SEQUENCE LISTING (25 sequences)

SEQ ID NO: 1 - protein, 20 aa, Artificial
  Synthetic histone peptide
ARTQTARKSTGGKAPRKQLK

SEQ ID NO: 2 - protein, 30 aa, Artificial
  Synthetic histone peptide
APRKQLATKAARKSAPSTGGVKKPHRYGGK

SEQ ID NO: 3 - protein, 12 aa, Artificial
  Synthetic histone peptide
IAQDFKTDLRFK

SEQ ID NO: 4 - protein, 20 aa, Artificial
  Synthetic histone peptide
KSAPSTGGVKKPHRYKPGTK

SEQ ID NO: 5 - protein, 26 aa, Artificial
  Synthetic histone peptide
TARKSTGGKAPRKQLATKAARKSAPK

SEQ ID NO: 6 - protein, 22 aa, Artificial
  Synthetic histone peptide
ARTQTARKSTGGKAPRKQLATK

SEQ ID NO: 7 - protein, 24 aa, Artificial
  Synthetic histone peptide
ARTQTARKSTGGKAPRKQLATKAA

SEQ ID NO: 8 - protein, 20 aa, Artificial
  Synthetic histone peptide
APRKQLATKAARKSAPSTGG

SEQ ID NO: 9 - protein, 21 aa, Artificial
  Synthetic histone peptide
PATGGVKKPHRYRPGTVALRK

SEQ ID NO: 10 - protein, 21 aa, Artificial
  Synthetic histone peptide
EDTNLCAIHAKRVTIMPKDIK

SEQ ID NO: 11 - protein, 21 aa, Artificial
  Synthetic histone peptide
APRKQLATKAARKSAPSTGGK

SEQ ID NO: 12 - protein, 21 aa, Artificial
  Synthetic histone peptide
AQDFKTDLRFQSAAIGALQEK

SEQ ID NO: 13 - protein, 16 aa, Artificial
  Synthetic histone peptide
MPKDIQLARRIRGERA

SEQ ID NO: 14 - protein, 32 aa, Artificial
  Synthetic histone peptide
ARTKQTARKSTGGKAPRKQLATKAARKSAPAT

SEQ ID NO: 15 - protein, 10 aa, Artificial
  Synthetic histone peptide
RRYQKSTELL

SEQ ID NO: 16 - protein, 15 aa, Artificial
  Synthetic histone peptide
ARTKQTARKSTGGKA

SEQ ID NO: 17 - protein, 23 aa, Artificial
  Synthetic histone peptide
SGRGKGGKGLGKGGAKRHRKVLR

SEQ ID NO: 18 - protein, 17 aa, Artificial
  Synthetic histone peptide
GKGGAKRHRKVLRDNIQ

SEQ ID NO: 19 - protein, 17 aa, Artificial
  Synthetic histone peptide
SGRGKQGGKARAKAKTR

SEQ ID NO: 20 - protein, 16 aa, Artificial
  Synthetic histone peptide
SAAKASQSRSAKAGLT

SEQ ID NO: 21 - protein, 16 aa, Artificial
  Synthetic histone peptide
ARAKAKTRSSRAGLQF

SEQ ID NO: 22 - protein, 11 aa, Artificial
  Synthetic histone peptide
GKKATQASQEY

SEQ ID NO: 23 - protein, 16 aa, Artificial
  Synthetic histone peptide
SAAKASAAAAAKAGLT

SEQ ID NO: 24 - protein, 17 aa, Artificial
  Synthetic histone peptide
SGRGKTGGKARAKAKSR

SEQ ID NO: 25 - protein, 24 aa, Artificial
  Synthetic histone peptide
PEPAKSAPAPKKGSKKAVTKAQKK

(19) United States
(12) Patent Application Publication
(10) Pub. No.: US 2013/0196867 A1
(43) Pub. Date: Aug. 1, 2013

(54) COMBINATORIAL POST-TRANSLATIONALLY-MODIFIED HISTONE PEPTIDES, ARRAYS THEREOF, AND METHODS OF USING THE SAME

(71) Applicant: The University of North Carolina at Chapel Hill, Chapel Hill, NC (US)

(72) Inventors: Brian D. Strahl, Chapel Hill, NC (US); Stephen M. Fuchs, Cambridge, MA (US); Krzysztof Krajewski, Cary, NC (US)

(73) Assignee: The University of North Carolina at Chapel Hill, Chapel Hill, NC (US)

(21) Appl. No.: 13/714,867

(22) Filed: Dec. 14, 2012

Related U.S. Application Data
(60) Provisional application No. 61/576,542, filed on Dec. 16, 2011.

(51) Int. Cl. G01N 33/68

(52) U.S. Cl. CPC G01N 33/6875
USPC 506/9; 506/18

(57) ABSTRACT

The present invention generally relates to combinatorial post-translationally-modified histone peptides and arrays thereof. The invention further relates to methods of using the same.

### RELATED APPLICATION DATA

This application claims the benefit of U.S. Provisional Patent Application Ser. No. 61/576,542, filed Dec. 16, 2011, the disclosure of which is incorporated herein by reference in its entirety

### STATEMENT OF FEDERAL SUPPORT

This invention was made with government support under National Institutes of Health (NIH) Grant No. GM085394-01. The United States government has certain rights to this invention.

### FIELD OF THE INVENTION

The present invention generally relates to combinatorial post-translationally-modified histone peptides and arrays thereof. The invention further relates to methods of using the same.

### BACKGROUND OF THE INVENTION

Protein posttranslational modifications (PTMs), such as phosphorylation, methylation, acetylation, and ubiquitination, regulate many processes, such as protein degradation, protein trafficking, and mediation of protein-protein interactions. Perhaps the best-studied PTMs are those found to be associated with histone proteins.

The enormous number of potential combinations of histone PTMs represents a major obstacle to our understanding of how PTMs regulate chromatin-templated processes, as well as to our ability to develop high-quality diagnostic tools for chromatin and epigenetic studies. A major limitation in exploring the full extent of the histone code has been the lack of a comprehensive library of modified histone peptides that can be used to rapidly and efficiently screen for effector proteins that bind to unique modification patterns.

The present invention addresses previous shortcomings in the art by providing combinatorial post-translationally-modified histone peptide, arrays thereof, and methods of using the same.

### SUMMARY OF THE INVENTION

A first aspect of the present invention comprises a plurality of synthetic histone peptides, wherein a portion of the synthetic histone peptides comprise at least one post-translational modification, and wherein a portion of the synthetic histone peptides are at least 21 amino acids in length.

A second aspect of the present invention comprises a peptide array comprising: a substrate comprising a surface; and a plurality of synthetic histone peptides immobilized on the substrate surface, wherein a portion of the synthetic histone peptides comprise at least one post-translational modification, and wherein a portion of the synthetic histone peptides are at least 21 amino acids in length.

Another aspect of the present invention comprises a method for determining the binding of a protein to a peptide comprising: providing a peptide array comprising: a substrate comprising a surface; and a plurality of synthetic histone peptides immobilized on the substrate surface, wherein a portion of the synthetic histone peptides comprise at least one post-translational modification, and wherein a portion of the synthetic histone peptides are at least 21 amino acids in length; applying a protein to the peptide array; and detecting binding of the protein to one or more synthetic histone peptides in the peptide array.

A further aspect of the present invention comprises a method for detecting the influence of neighboring post-translational modifications on protein binding comprising: providing a peptide array comprising: a substrate comprising a surface; and a plurality of synthetic histone peptides immobilized on the substrate surface, the plurality of synthetic histone peptides comprising peptides with no post-translational modifications, peptides with one post-translational modification, and peptides with more than one post-translational modification, wherein a portion of the synthetic histone peptides are at least 21 amino acids in length; applying a protein to the peptide array; detecting binding of the protein to one or more synthetic histone peptides in the peptide array; and comparing the sequences of the synthetic histone peptides bound to the protein, thereby detecting the influence of neighboring post-translational modifications on protein binding.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1A shows exemplary peptides synthesized and the possible side-chain modifications (in single or combinatorial fashion) indicated for each amino acid.

FIG. 1B shows the peptide array surface, which comprised streptavidin-coated glass slides that were spotted with a library of histone peptides containing different combinations of posttranslational modifications. Biotin-fluorescein was mixed with the peptides and used as an internal control for spotting efficiency.

FIG. 1C shows a fluorescent image from a sample array. Positive binding interactions are shown as light grey spots where only the printing control (medium grey) is visible for negative interactions.

FIG. 2 shows a heat map of all experimental antibody data. Data was normalized to the strongest interaction plotted on a scale from 0 to 1 with 1 (light grey) being the most significant.

FIG. 3 shows the results of two independent arrays consisting of 24 independent spots for each peptide, which are depicted as heat maps of the normalized mean intensity and plotted on a scale from 0 to 1, with 1 (light grey) being the most significant. FIG. 3(A) shows the interactions of H3K4- and H3K79-specific antibodies with methylated peptides derived from the N terminus of histone H3. FIG. 3(B) shows the recognition of histone H3 acetyllysine peptides by H3K14ac antibodies. FIG. 3(C) shows the western blot of yeast whole-cell extract probed with H3K14ac antibody preincubated with various concentrations of histone H3 peptides. FIG. 3(D) shows the alignment of sequence surrounding H3K14 and H3K16.

FIG. 4 shows a comparison of H3K4 methyllysine-specific antibodies for different methylation states (H3K4me1—Millipore 07-436, H3K4me2—Active Motif 39142, H3K4me3—Active Motif 39160). Data are plotted as the mean with SEM for the indicated peptide from a single array. The results of two independent arrays are shown. Differences in intensities were compared using two-way ANOVA analyses and confidence intervals (* 95% and *** 99.9%) are indicated for individual comparisons.

FIG. 5 shows the results of two independent arrays consisting of 24 independent spots for each peptide, which are depicted as heat maps of the normalized mean intensity and plotted on a scale from 0 to 1, with 1 (light grey) being the most significant. FIG. 5(A) shows a heat map depicting the effects of neighboring modifications on H3K4me3-specific antibody recognition. 3ac=K9ac, K14ac, and K18ac. FIG. 5(B) shows the recognition of H3S10 phosphorylation by mono- and dual-specific PTM antibodies. FIG. 5(C) shows a bar graph of data from FIG. 5(B), Differences in intensities were compared using two-way analyses of variance, and confidence intervals (99% [**]) are indicated for individual comparisons.

FIG. 6 shows chromatin-associating domain binding to histone peptide arrays. FIG. 6(A) (top) shows a heat map of RAG2 PHD domain binding to histone H3 peptides and (bottom) shows a molecular representation of the RAG2 PHD domain binding to an H3K4me3-containing peptide (PDB accession 2V83). FIG. 6(B) (top) shows a heat map of RAG2 PHD-Bromo domain binding to histone H3 peptides and (bottom) shows a molecular representation of the BPTF PHD domain binding to an H3K4me3-containing peptide (PDB accession 2F6J). FIG. 6(C) (top) shows a heat map of CHD1 chromodomain binding to histone H3 peptides and (bottom) shows a molecular representation of the CHD1 chromodomain binding to an H3K4me3-containing peptide (PDB accession 2B2W). All models were constructed using PyMol software.

FIG. 7 shows the effect of neighboring acetylation on BPTF binding, Data are plotted as the mean with SEM for the indicated peptide from a single array. Differences in intensities were compared using two-way ANOVA analyses and confidence intervals (* 95%, ** 99% and *** 99.9%) are indicated for comparisons to the H3K4me3 peptide with no other modifications (left).

FIG. 8 shows scatter plots comparing the two arrays for (a) RAG2, (b) BPTF, and (c) CHD1.

FIG. 9A-C shows UHRF1 TTD binds H3K9me regardless of neighboring H3S10ph. FIG. 9A shows the peptide microarray analysis of the indicated H3K9 effector domains, Results of at least two arrays are presented as heat maps of normalized mean intensities on a scale from 0 (black; undetectable binding) to 1 (light grey; strong binding). FIG. 9B shows the western blot following in-solution peptide pulldowns with the indicated domains. FIG. 9C shows the fluorescence polarization binding assays of H3K9me3 peptides with the indicated protein domains in the absence (circle) or presence (square) of H3S10p. Error is represented as ±s.d. for three independent experiments. The y-axis is on the same scale for all plots.

### DETAILED DESCRIPTION OF THE INVENTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976); see also MPEP §2111.03. Thus, the term “consisting essentially of” as used herein should not be interpreted as equivalent to “comprising.”

The term “about,” as used herein when referring to a measurable value such as an amount or concentration (e.g., the amount of a peptide) and the like, is meant to encompass variations of ±10%, ±5%, ±1%, ±0.5%, or even ±0.1% of the specified amount. A range provided herein for a measureable value may include any other range and/or individual value therein.

The present invention comprises, consists essentially of, or consists of a synthetic histone peptide. A “synthetic histone peptide” is a peptide that is synthetically produced and comprises an amino acid sequence similar to a naturally occurring histone amino acid sequence. Histones are known in the art and as those of skill in the art will appreciate, the amino acid sequence of a histone can be obtained by known methods. For example, amino acid sequences useful to the present invention can be obtained through publicly available databases, such as the National Center for Biotechnology Information (NCBI) database. Exemplary histones include, but are not limited to, H1, H2A, H2B, H3, H4, H5, or any combination thereof. In some embodiments of the present invention, the amino acid sequence of a synthetic histone peptide can be similar to one or more, such as 2, 3, 4, or more, naturally occurring histone amino acid sequences. Synthetic histone peptides of the present invention can be synthesized using methods known in the art, such as, but not limited to chemical peptide synthesis methods, including using an automated peptide synthesizer. The amino acid sequence of a synthetic histone peptide can be similar to a N-terminal tail of a histone, a C-terminal tail of a histone, an internal region of a histone, or any combination thereof. In some embodiments of the present invention, the amino acid sequence of a synthetic histone peptide is similar to a N-terminal tail of a histone or a C-terminal tail of a histone.

A synthetic histone peptide can comprise from 10 to 40 amino acids in length or any range therein, such as, but not limited to, from 15 to 35 amino acids or 20 to 30 amino acids. In particular embodiments of the present invention, a synthetic histone peptide is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length, or any range therein. In certain embodiments of the present invention, a synthetic histone peptide is at least 21 amino acids in length, e.g., at least 22, 23, 24, 25, 26, 27, 28, 29, 30, 31, 32, 33, 34, 35, 36, 37, 38, 39, or 40 amino acids in length, or any range therein.

“Similar” as used herein in reference to the amino acid sequence of a synthetic histone peptide and a naturally occurring histone amino acid sequence refers to a synthetic histone amino acid sequence that is at least about 70%, 75%, 80%, 85%, 90%, 95%, 97%, 98%, 99%, or 100% identical to a naturally occurring histone amino acid sequence. In some embodiments of the present invention, the amino acid sequence of a synthetic histone peptide is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or any range therein, identical to a naturally occurring histone amino acid sequence. According to some embodiments of the present invention, a section or piece of a synthetic histone peptide (e.g., 5 to 25 consecutive amino acids or any range therein) can be similar to one or more naturally occurring histone amino acid sequences.

A synthetic histone peptide of the present invention can be “similar” to a naturally occurring histone amino acid sequence in that during the design and/or preparation of the synthetic histone peptide, a naturally occurring histone amino acid sequence is used as a template and/or model amino acid sequence, but one or more amino acids in the naturally occurring histone amino acid sequence are changed and/or modified to comprise a post-translational modification, a different amino acid, and/or an amino acid derivative. “Amino acid derivative” as used herein, refers to an amino acid substituted with one or more substituents. Exemplary substituents include, but are not limited to, alkyl, lower alkyl, halo, haloalkyl, alkenyl, alkynyl, cycloalkyl, cycloalkylalkyl, heterocyclo, heterocycloalkyl, aryl, arylalkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silylalkyl, silyloxy, boronyl, modified lower alkyl, and any combination thereof. Exemplary amino acid derivatives include, but are not limited to, alanine methyl ester, valine ethyl ester, phenylalainamide, N-acetyl-tyrosine, and O-benzyl-tyrosine. In some embodiments of the present invention, 1, 2, 3, 4, 5, 6, 7, 8, 9, 10 or more amino acids in a naturally occurring histone amino acid sequence are changed and/or modified to produce a synthetic histone peptide of the present invention. In particular embodiments of the present invention, the change and/or modification comprises one or more post-translational modifications.

“Post-translational modification” as used herein refers to a chemical modification to an amino acid. In naturally occurring peptides and/or proteins post-translational modifications occur after the peptide/protein is translated. Thus, as those skilled in the art will recognize, a naturally occurring histone amino acid sequence can comprise one or more post-translational modifications. Post-translational modifications include, but are not limited to, phosphorylation, methylation (e.g., lysine methylation (mono-, di-, or trimethylation) and arginine methylation (mono, asymmetric dimethylation, or symmetric dimethylation)), acetylation, ubiquitination, myristoylation, palmitoylation, isoprenylation, prenylation, acylation, glycosylation, hydroxylation, iodination, oxidation, sulfation, selenoylation, SUMOylation, citrullination, deamidation, carbamylation, ADP-ribosylation, lysine crotonylation, formylation, propionyllysine, butyryllysine, or any combination thereof. One or more post-translational modifications of a synthetic histone peptide of the present invention can comprise changing and/or modifying an amino acid during and/or after the synthesis of the synthetic histone peptide.

In some embodiments of the present invention, a synthetic histone peptide comprises one or more post-translational modifications, such as 2, 3, 4, 5, 6, 7, 8, 9, or more post-translational modifications. When more than one post-translational modification is present in a synthetic histone peptide of the present invention, the post-translational modifications can be the same and/or different. For example, the post-translational modifications can be the same type of post-translation modification (e.g., methylation) on different amino acids, which can be the same (e.g., the two modified amino acids are lysine) or different (e.g., the two modified amino acids are lysine and serine). When the two or more post-translational modifications are different types (e.g., methylation and acetylation), the modifications are on different amino acids, which can be the same or different. In certain embodiments of the present invention, two or more different types of post-translational modifications, such as 2, 3, 4, 5, 6, 7, 8, 9, or more, are present in a synthetic histone peptide of the present invention.

The one or more post-translational modifications in a synthetic histone peptide of the present invention can be the same as and/or different than the post-translational modifications found in a naturally occurring histone amino acid sequence. For example, a post-translational modification can be the same type of post-translation modification (e.g., methylation) on a specific amino acid in both a naturally occurring histone amino acid sequence and synthetic histone peptide sequence. A post-translational modification in a synthetic histone peptide of the present invention can be different compared to a post-translation modification on a specific amino acid in a naturally occurring histone amino acid sequence (e.g., the modification is methylation on a specific lysine in a synthetic histone peptide and acetylation on the corresponding lysine in a naturally occurring histone amino acid sequence). Similarly, a synthetic histone peptide of the present invention can comprise an amino acid that is not post-translationally modified (i.e., an unmodified amino acid) as it may be found in a naturally occurring histone amino acid sequences (i.e., a naturally occurring histone amino acid sequence comprises a post-translational modification on a specific amino acid and a similar synthetic histone peptide does not comprise that modification, but rather is an unmodified amino acid). Exemplary synthetic histone peptides of the present invention include, but are not limited to, those shown in Tables 1 and 2.

TABLE 1

| | Similar | |
|---|---|---|
| Peptide | Histone | |
| # | Sequence | Peptide Amino Acid Sequence |
| | | H3 PEPTIDES |
| P1 | H3 1-20 | ARTKQTARKSTGGKAPRKQL-K(Biot)-NH2 |
| P2 | H3 1-20 | ARTKQTARKSTGGK(Ac)APRKQL-K(Biot)-NH2 |
| P3 | H3 1-20 | ARTKQTARK(Ac)STGGKAPRKQL-K(Biot)-NH2 |
| P4 | H3 1-20 | ARTK(Ac)QTARKSTGGKAPRKQL-K(Biot)-NH2 |
| P5 | H3 1-20 | ARTK(Ac)QTARKSTGGK(Ac)APRKQL-K(Biot)-NH2 |
| P6 | H3 1-20 | ARTKQTARK(Ac)STGGK(Ac)APRKQL-K(Biot)-NH2 |
| P7 | H3 1-20 | ARTK(Ac)QTARK(Ac)STGGKAPRKQL-K(Biot)-NH2 |
| P8 | H3 1-20 | ARTK(Ac)QTARK(Ac)STGGK(Ac)APRKQL-K(Biot)-NH2 |
| P9 | H3 1-20 | ARTKQTARKSTGGKAPRKQL-K(Biot)-NH2 |
| P10 | H3 1-20 | ARTKQTARKSTGGKAPRK(Ac)QL-K(Biot)-NH2 |
| P11 | H3 1-20 | ARTKQTARKSTGGK(Ac)APRK(Ac)QL-K(Biot)-NH2 |
| P12 | H3 1-20 | ARTKQTARK(Ac)STGGKAPRK(Ac)QL-K(Biot)-NH2 |
| P13 | H3 1-20 | ARTK(Ac)QTARKSTGGKAPRK(Ac)QL-K(Biot)-NH2 |
| P14 | H3 1-20 | ARTKQTARK(Ac)STGGK(Ac)APRK(Ac)QL-K(Biot)-NH2 |
| P15 | H3 1-20 | ARTK(Ac)QTARKSTGGK(Ac)APRK(Ac)QL-K(Biot)-NH2 |
| P16 | H3 1-20 | ARTK(Ac)QTARK(Ac)STGGKAPRK(Ac)QL-K(Biot)-NH2 |
| P17 | H3 1-20 | ARTK(Ac)QTARK(Ac)STGGK(Ac)APRK(Ac)QL-K(Biot)-NH2 |
| P18 | H3 1-20 | ARTK(Me3)QTARKSTGGKAPRKQL-K(Biot)-NH2 |
| P19 | H3 1-20 | ARTK(Me3)QTARK(Ac)STGGKAPRKQL-K(Biot)-NH2 |
| P20 | H3 1-20 | ARTK(Me3)QTARKSTGGK(Ac)APRKQL-K(Biot)-NH2 |
| P21 | H3 1-20 | ARTK(Me3)QTARKSTGGICAPRK(Ac)QL-K(Biot)-NH2 |
| P22 | H3 1-20 | ARTK(Me3)QTARK(Ac)STGGK(Ac)APRKQL-K(Biot)-NH2 |
| P23 | H3 1-20 | ARTK(Me3)QTARK(Ac)STGGKAPRK(Ac)QL-K(Biot)-NH2 |
| P24 | H3 1-20 | ARTK(Me3)QTARKSTGGK(Ac)APRK(Ac)QL-K(Biot)-NH2 |
| P25 | H3 1-20 | ARTK(Me.3)QTARK(Ac)STGGK(Ac)APRK(Ac)QL-K(Biot)-NH2 |
| P26 | H3 1-20 | ARpTK(Me3)QTARK(Ac)STGGK(Ac)APRK(Ac)QL-K(Biot)-NH2 |
| P27 | H3 1-20 | ARpTK(Me3)QTARKSTGGKAPRKQL-K(Biot)-NH2 |
| P28 | H3 1-20 | AR(Me2a)pTK(Me3)QTARK(Ac)STGGK(Ac)APRK(Ac)QL-K(Biot)-NH2 |
| P29 | H3 1-20 | AR(Me2a)pTK(Me3)QTARKSTGGKAPRKQL-K(Biot)-NH2 |
| P30 | H3 1-20 | AR(Me2a)TK(Me3)QTARKSTGGKAPRKQL-K(Biot)-NH2 |
| P31 | H3 1-20 | 5-Fam-ARTKQTARKSTGGKAPRKQL-K(Biot)-NH2 |
| P32 | H3 1-20 | ARTK(Me2)QTARKSTGGKAPRKQL-K(Biot)-NH2 |
| P33 | H3 1-20 | ARTK(Me2)QTARK(Ac)STGGK(Ac)APRK(Ac)QL-K(Biot)-NH2 |
| P34 | H3 1-20 | ARTK(Me)Q TARKSTGGKAPRKQL-K(Biot)-NH2 |
| P35 | H3 1-20 | ARTK(Me)QTARK(Ac)STGGK(Ac)APRK(Ac)QL-K(Biot)-NH2 |
| P36 | H3 1-20 | ARTKQTARKpSTGGKAPRKQL-K(Biot)-NH2 |
| P37 | H3 1-20 | ARTK(Ac)QTARK(Ac)pSTGGK(Ac)APRK(Ac)QL-K(Biot)-NH2 |
| P38 | H3 1-20 | ARTK(Me3)QTARKpSTGGKAPRKQL-K(Biot)-NH2 |
| P39 | H3 1-20 | ARTK(Me3)QTARK(Ac)pSTGGK(Ac)APRK(Ac)QL-K(Biot)-NH2 |
| P40 | H3 1-20 | AR(Me2a)TK(Me3)QTARKpSTGGKAPRKQL-K(Biot)-NH2 |
| P41 | H3 1-20 | AR(Me2a)TK(Me3)QTARK(Ac)pSTGGK(Ac)APRK(Ac)QL-K(Biot)-NH2 |
| P42 | H3 1-20 | ARTKQTARK(Me3)STGGKAPRKQL-K(Biot)-NH2 |
| P43 | H3 1-20 | ARTK(Ac)QTARK(Me3)STGGK(Ac)APRK(Ac)QL-K(Biot)-NH2 |
| P44 | H3 1-20 | ARTK(Me2)QTARK(Ac)STGGKAPRK(Ac)QL-K(Biot)-NH2 |
| P45 | H3 1-20 | ARTK(Me)QTARK(Ac)STGGKAPRK(Ac)QL-K(Biot)-NH2 |
| P46 | H3 1-20 | ARTKQTARK(Me3)TGGKAPRKQL-K(Biot)-NH2 |
| P47 | H3 1-20 | AR(Me2a)TKQTARKSTGGKAPRKQL-K(Biot)-NH2 |
| P48 | H3 1-20 | AR(Me2a)TK(Ac)QTARK(Ac)STGGK(Ac)APRK(Ac)QL-K(Biot)-NH2 |
| P50 | H3 1-20 | AR(Me2a)TK(Me3)QTARK(Ac)STGGK(Ac)APRK(Ac)QL-K(Biot)-NH2 |
| P51 | H3 1-20 | AR(Me)TK(Me3)QTARKSTGGKAPRKQL-K(Biot)-NH2 |
| P52 | H3 1-20 | AR(Me)TK(Me3)QTARK(Ac)STGGK(Ac)APRK(Ac)QL-K(Biot)-NH2 |
| P53 | H3 1-20 | ACitTKQTARKSTGGKAPRKQL-K(Biot)-NH2 |
| P54 | H3 1-20 | ACitTK(Me3)QTARKSTGGKAPRKQL-K(Biot)-NH2 |
| P55 | H3 1-20 | ACitTK(Me3)QTARK(Ac)STGGK(Ac)APRK(Ac)QL-K(Biot)-NH2 |
| P56 | H3 1-20 | ACitTK(Ac)QTARK(Ac)STGGK(Ac)APRK(Ac)QL-K(Biot)-NH2 |
| P57 | H3 1-20 | ARpTKQTARKSTGGKAPRKQL-Peg-K(Biot)-NH2 |
| P60 | H3 1-20 | AR(Me2a)TK(Me2)QTARKSTGGKAPRKQL-K(Biot)-NH2 |
| P61 | H3 1-20 | AR(Me2s)TK(Me2)QTARKSTGGKAPRKQL-K(Biot)-NH2 |
| P62 | H3 1-20 | AR(Me)TK(Me2)QTARKSTGGKAPRKQL-K(Biot)-NH2 |
| P63 | H3 1-20 | ACitTK(Me2)QTARKSTGGKAPRKQL-K(Biot)-NH2 |
| P65 | H3 1-20 | ARTK(N3)QTARKSTGGKAPRKQL-K(Biot)-NH2 |
| P89 | H3 1-20 | ARTK(Me3)QTAR(Me2s)K(Me3)STGGKAPRKQL-K(Biot)-NH2 |
| P90 | G H3 15-43 | Ac-APRK18QLATK23AARK27SAPSTGGVK36K37PHRYGGK(Biot)-NH2 |
| P91 | G H3 15-43 | Ac-APRK(Me3)QLATKAARKSAPSTGGVKKPHRY-GG-K(Biot)-NH2 |
| P93 | G H3 15-43 | Ac-APRKQLATKAARKSAPSTGGVK(Me3)KPHRY-GG-K(Biot)-NH2 |
| P95 | G H3 15-43 | Ac-APRK(Me3)QLATKAARKSAPSTGGVK(Me3)KPHRY-GG-K(Biot)-NH2 |
| P96 | H3 1-20 | ARTK(Me3)QTAR(Me2a)K(Me3)STGGKAPRKQL-K(Biot)-NH2 |
| P100 | H3 74-84 | Ac-IAQDFKTDLRF-Peg-K(Biot)-NH2 |
| P101 | H3 74-84 | Ac-IAQDFK(Me3)TDLRF-Peg-K(Biot)-NH2 |
| P102 | H3 74-84 | Ac-IAQDFK(Me2)TDLRF-Peg-K(Biot)-NH2 |
| P103 | H3 74-84 | Ac-IAQDFK(Me)TDLRF-Peg-K(Biot)-NH2 |
| P104 | H3 74-84 | IAQDFKTDLRF-Peg-K(Biot)-NH2 |
| P105 | H3 74-84 | Ac-IAQDFK(Me2)pTDLRF-Peg-K(Biot)-NH2 |
| P106 | H3 74-84 | Ac-IAQDFKpTDLRF-Peg-K(Biot)-NH2 |
| P120 | H3 27-45 | KSAPSTGGVK(Me3)KPHRYKPGT-G-K(Biot)-NH2 |
| P121 | H3 27-45 | KSAPSTGGVK(Me2)KPHRYKPGT-GG-K(Biot)-NH2 |
| P122 | H3 27-45 | KSAPSTGGVK(Me)KPHRYKPGT-GG-K(Biot)-NH2 |
| P123 | H3 27-45 | KSAPSTGGVK(Ac)KPHRYKPGT-GG-K(Biot)-NH2 |
| P124 | H3 27-45 | KSAPSTGGVKKPHRYKPGT-GG-K(Biot)-NH2 |
| P125 | H3 1-20 | ARpTKQTARKSTGGKAPRKQL-K(Biot)-NH2 |
| P126 | H3 27-45 | KSAPSTGGVK(Me)KPHRYKPGT-G-K(Biot)-NH2 |
| P127 | H3 27-45 | KSAPpSTGGVK(Me3)KPHRYKPGT-G-K(Biot)-NH2 |
| P128 | H3 27-45 | KSAPpSTGGVKKPHRYKPGT-G-K(Biot)-NH2 |
| P129 | H3 6-30 | Ac-TARK(Me2)STGGKAPRKQLATKAARK(Me2)SAP-Peg-K(Biot)-NH2 |
| P132 | H3 1-20 | ARTK(Me3)QTARK(Me3)STGGKAPRKQL-K(Biot)-NH2 |
| P133 | H3 1-20 | ARTKQTARK(Me2)STGGKAPRKQL-K(Biot)-NH2 |
| P134 | H3 1-20 | ARTKQTARK(Me)STGGKAPRKQL-K(Biot)-NH2 |
| P135 | H3 1-20 | ARTK(Me)QTARKSTGGKAPRK(Ac)QL-Peg-Biot |
| P136 | H3 1-20 | ARTKQTARKSpTGGKAPRKQL-Peg-Biot |
| P137 | H3 1-20 | ARTKQTARKSTGGKAPRK(Me3)QL-K(Biot)-NH2 |
| P138 | H3 1-20 | ARTKQTARKSTGGKAPRK(Me2)QL-K(Biot)-NH2 |
| P139 | H3 1-20 | ARTKQTARKSTGGKAPRK(Me)QL-K(Biot)-NH2 |
| P140 | H3 1-20 | ARTKQTAR(Me)KSTGGKAPRKQL-Peg-Biot |
| P141 | H3 1-20 | ARTKQTAR(Me2a)KSTGGKAPRKQL-Peg-Biot |
| P142 | H3 1-20 | ARTKQTAR(Me2s)KSTGGKAPRKQL-Peg-Biot |
| P144 | H3 1-20 | ARTKQTARK(Ac)pSTGGKAPRKQL-K(Biot)-NH2 |
| P145 | H3 1-20 | ARTKQTARK(Me3)pSTGGKAPRKQL-K(Biot)-NH2 |
| P146 | H3 1-20 | ARTKQTARK(Me2)pSTGGKAPRKQL-K(Biot)-NH2 |
| P147 | H3 1-20 | ARTKQTARK(Me)pSTGGKAPRKQL-K(Biot)-NH2 |
| P148 | H3 1-20 | ARTK(Me3)QTARK(Ac)pSTGGKAPRKQL-K(Biot)-NH2 |
| P149 | H3 1-22 | ARTKQTARKSTGGKAPR(Me2a)KQLAT-K(Biot)-NH2 |
| P150 | H3 1-22 | ARTKQTARKSTGGKAPR(Me2s)KQLAT-K(Biot)-NH2 |
| P151 | H3 1-22 | ARTKQTARKSTGGKAPR(Me)KQLAT-K(Biot)-NH2 |
| P152 | H3 1-22 | ARTKQTARK(Ac)STGGK(Ac)APR(Me2a)K(Ac)QLAT-K(Biot)-NH2 |
| P153 | H3 1-22 | ARTKQTARK(Ac)STGGK(Ac)APR(Me2s)K(Ac)QLAT-K(Biot)-NH2 |
| P154 | H3 1-22 | ARTKQTARK(Ac)STGGK(Ac)APR(Me)K(Ac)QLAT-K(Biot)-NH2 |
| P155 | H3 1-20 | ARTKQTARKSTGGK(Me2)APRKQL-Peg-Biot |
| P156 | H3 1-20 | ARTKQTARKSTGG K(Me3)APRKQL-Peg-Biot |
| P157 | H3 1-20 | AR(Me2s)TK(Me3)QTARKSTGGKAPRKQL-K(Biot)-NH2 |
| P158 | H3 1-25 | ARTKQTARKSTGGKAPRK(Ac)QLATKAA-Peg-Biot |
| P159 | H3 1-25 | ARTKQTARKSTGGKAPR(Me2a)KQLATKAA-Peg-Biot |
| P160 | H3 1-25 | ARTKQTARKSTGGKAPR(Me2a)K(Ac)QLATKAA-Peg-Biot |
| P161 | H3 1-25 | ARTKQTARKSTGGKAPRKQLATKAA-Peg-Biot |
| P162 | H3 1-20 | ARTKQpTARKSTGGKAPRKQL-K(Biot)-NH2 |
| P163 | H3 1-20 | ARTK(Me3)QpTARKSTGGKAPRKQL-K(Biot)-NH2 |
| P164 | H3 1-20 | ARTK(Me2)QpTARKSTGGKAPRKQL-K(Biot)-NH2 |
| P165 | H3 1-20 | ARTKQpTARK(Ac)STGGK(Ac)APRK(Ac)QL-K(Biot)-NH2 |
| P166 | H3 1-20 | ARTK(Me3)QpTARK(Ac)STGGK(Ac)APRK(Ac)QL-K(Biot)-NH2 |
| P167 | H3 1-20 | ARTK(Me2)QpTARK(Ac)STGGK(Ac)APRK(Ac)QL-K(Biot)-NH2 |
| P174 | H3 1-20 | AR(Me2s)TK(Me3)QTARK(Ac)STGGK(Ac)APRK(Ac)QL-K(Biot)-NH2 |
| P178 | H3 1-20 | ARTKQTAR(Me)K(Me3)STGGKAPRKQL-K(Biot)-NH2 |
| P179 | H3 1-20 | ARTKQTAR(Me)K(Me2)STGGKAPRKQL-K(Biot)-NH2 |
| P180 | H3 1-20 | ARTKQTAR(Me2a)K(Me3)STGGKAPRKQL-K(Biot)-NH2 |
| P181 | H3 1-20 | ARTKQTAR(Me2a)K(Me2)STGGKAPRKQL-K(Biot)-NH2 |
| P182 | H3 1-20 | ARTKQTAR(Me2a)K(Me)STGGKAPRKQL-K(Biot)-NH2 |
| P183 | H3 1-20 | ARTKQTAR(Me2s)K(Me3)STGGKAPRKQL-K(Biot)-NH2 |
| P184 | H3 1-20 | ARTKQTAR(Me2s)K(Me2)STGGKAPRKQL-K(Biot)-NH2 |
| P185 | H3 1-20 | ARTKQTAR(Me2s)K(Me)STGGKAPRKQL-K(Biot)-NH2 |
| P186 | H3 1-20 | ARTK(Ac)QTARK(Me2)STGGK(Ac)APRK(Ac)QL-K(Biot)-NH2 |
| P187 | H3 1-20 | ARTK(Ac)QTARK(Me)STGGK(Ac)APRK(Ac)QL-K(Biot)-NH2 |
| P195 | H3 15-34 | Ac-APRK18QLATK23AARK(Me3)27SAPSTGG-Peg-Biot |
| P196 | H3 15-34 | Ac-APRK18QLATK23AARK(Me2)27SAPSTGG-Peg-Biot |
| P197 | H3 15-34 | Ac-APRK18QLATK23AARK(Me)27SAPSTGG--Peg-Biot |
| P198 | H3 15-34 | Ac-APRK18QLATK23AAR(Me2a)K(Me3)27SAPSTGG--Peg-Biot |
| P199 | H3 15-34 | Ac-APRK18QLATK23AAR(Me2a)K(Me2)27SAPSTGG-Peg-Biot |
| P200 | H3 15-34 | Ac-APRK18QLATK23AARR(Me2a)K(Me)27SAPSTGG-Peg-Biot |
| P202 | H3 15-34 | Ac-APRKQLATKAARKSAPATGG-Peg-K(Biot)-NH2 |
| P203 | H3 30-49 | Ac-PATGGVKKPHRYRPGTVALR-Peg-K(Biot)-NH2 |
| P208 | H3 105-124 | Ac-EDTNLCAIHAKRVTIMPKDI-Peg-K(Biot)-NH2 |
| P209 | H3.3 15-34 | Ac-APRKQLATKAARKSAPSTGG-Peg-K(Biot)-NH2 |
| P211 | H3.3 75-94 | Ac-AQDFKTDLRFQSAAIGALQE-Peg-K(Biot)-NH2 |
| P213 | H3 120-135 | (Biot)Peg-MPKDIQLARRIRGERA-OH |
| P220 | H3 1-20 | ARTKQpTARK(Me3)STGGKAPRKQL-K(Biot)-NH2 |
| P221 | H3 1-20 | ARTKQpTAR(Me2a)K(Me3)STGGKAPRKQL-K(Biot)-NH2 |
| P222 | H3 1-20 | ARpTK(Me2)QTARKSTGGKAPRKQL-K(Biot)-NH2 |
| P223 | H3 1-20 | ARpTK(Me)QTARKSTGGKAPRKQL-K(Biot)-NH2 |
| P224 | H3 15-34 | Ac-APRK18QLATK23AAR(Me2a)K27SAP STGG-Peg-Biot |
| P225 | H3 15-34 | Ac-APRK18QLATK23AARK(Me3)27pSAPSTGG-Peg-Biot |
| P226 | H3 15-34 | Ac-APRKI8QLATK23AARK(Me2)27pSAPSTGG-Peg-Biot |
| P227 | H3 15-34 | Ac-APRK18QLATK23AARK(Me)27pSAPSTGG-Peg-Biot |
| P229 | H3 1-20 | ARTK(Ac)QTARK(Me3)STGGKAPRKQL-K(Biot)-NH2 |
| P237 | H3 1-32 | ARTKQTARK(Me2)STGGKAPRKQLATKAARKSAPAT-Peg-Biot |
| P238 | H3 1-32 | ARTKQTARK(Me2)STGGKAPRKQLATKAARK(Me)SAPAT-Peg-Biot |
| P239 | H3 1-32 | ARTKQTARKSTGGKAPRKQLATKAARK(Me)SAPAT-Peg-Biot |
| P253 | H3 52-61 | Ac-RRYQK56STELL-Peg-Biot |
| P254 | H3 52-61 | Ac-RRYQK(Ac)STELL-Peg-Biot |
| P255 | H3 52-61 | Ac-RRYQK(Me3)STELL-Peg-Biot |
| P258 | H3 1-15 | ARTKQTARK(Me2)STGGKA-Peg-Biot |
| P259 | H3 1-15 | ARTK(Me2)QTARK(Me2)STGGKA-Peg-Biot |
| P260 | H3 1-15 | ARTK(Me)QTARK(Me2)STGGKA-Peg-Biot |
| P264 | H3 1-15 | ARTK(Me3)QTARK(Me2)STGGKA-Peg-Biot |
| P265 | H3 1-15 | ARTAQTARK(Me2)STGGKA-Peg-Biot |
| P273 | H3 52-61 | Ac-RRYQK(Me)STELL-Peg-Biot |
| P275 | H3 52-61 | Ac-RRYQ K(Me2)STELL-Peg-Biot |
| | | H4 PEPTIDES |
| P58 | H4 1-23 | Ac-SGRGK5GGKGLGKGGAKRHRKVLR-Peg-Biot |
| P59 | H4 1-23 | Ac-SGRGK(Ac)GGK(Ac)GLGK(Ac)GGAK(Ac)RHRKVLR-Peg-Biot |
| P66 | H4 1-23 | Ac-SGRGK(Ac)GGKGLGKGGAKRHRKVLR-Peg-Biot |
| P67 | H4 1-23 | Ac-SGRGKGGK(Ac)GLGKGGAKRHRKVLR-Peg-Biot |
| P68 | H4 1-23 | Ac-SGRGKGGKGLGK(Ac)GGAKRHRKVLR-Peg-Biot |
| P69 | H4 1-23 | Ac-SGRGKGGKGLGKGGAK(Ac)RHRKVLR-Peg-Biot |
| P70 | H4 1-23 | Ac-SGRGK(Ac)GGKGLGK(Ac)GGAKRHRKVLR-Peg-Biot |
| P71 | H4 1-23 | Ac-SGRGKGGK(Ac)GLGKGGAK(Ac)RHRKVLR-Peg-Biot |
| P72 | H4 1-23 | Ac-SGRGK(Ac)GGK(Ac)GLGK(Ac)GGAKRHRKVLR-Peg-Biot |
| P73 | H4 1-23 | Ac-SGR(Me2a)GKGGKGLGKGGAKRHRKVLR-K(Biot)-NH2 |
| P74 | H4 1-23 | Ac-SGR(Me2s)GKGGKGLGKGGAKRHRKVLR-K(Biot)-NH2 |
| P75 | H4 1-23 | Ac-SGR(Me)GKGGKGLGKGGAKRHRKVLR-K(Biot)-NH2 |
| P76 | H4 1-23 | Ac-pSGR(Me2a)GKGGKGLGKGGAKRHRKVLR-K(Biot)-NH2 |
| P77 | H4 1-23 | Ac-pSGR(Me2s)GKGGKGLGKGGAKRHRKVLR-K(Biot)-NH2 |
| P78 | H4 1-23 | Ac-pSGR(Me)GKGGKGLGKGGAKRHRKVLR-K(Biot)-NH2 |
| P79 | H4 1-23 | Ac-SGR(Me2a)GK(Ac)GGK(Ac)GLGK(Ac)GGAK(Ac)RHRK(Ac)VLR-K(Biot)-NH2 |
| P80 | H4 1-23 | Ac-SGR(Me2s)GK(Ac)GGK(Ac)GLGK(Ac)GGAK(Ac)RHRK(Ac)VLR-K(Biot)-NH2 |
| P81 | H4 1-23 | Ac-SGR(Me)GK(Ac)GGK(Ac)GLGK(Ac)GGAK(Ac)RHRK(Ac)VLR-K(Biot)-NH2 |
| P82 | H4 11-27 | Ac-GKGGAKRHRK(Me3)VLRDNIQ-Peg-Biot |
| P83 | H4 11-27 | Ac-GKGGAKRHRK(Me2)VLRDNIQ-Peg-Biot |
| P84 | H4 11-27 | Ac-GKGGAKRHRK(Me)VLRDNIQ-Peg-Biot |
| P85 | H4 11-27 | Ac-GK(Ac)GGAK(Ac)RHRK(Me3)VLRDNIQ-Peg-Biot |
| P86 | H4 11-27 | Ac-GK(Ac)GGAK(Ac)RHRK(Me2)VLRDNIQ-Peg-Biot |
| P87 | H4 11-27 | Ac-GK(Ac)GGAK(Ac)RHRK(Me)VLRDNIQ-Peg-Biot |
| P88 | H4 11-27 | Ac-GK(Ac)GGAK(Ac)RHRKVLRDNIQ-Peg-Biot |
| P99 | H4 11-27 | Ac-GKGGAKRHRKVLRDNIQ-Peg-Biot |
| P350 | H4 1-23 | Ac-SGR(Me2a)GK(Ac)GGKGLGKGGAKRHRKVLR-K(Biot)-NH2 |
| P351 | H4 1-23 | SGRGKGGKGLGKGGAKRHRKVLR-Peg-Biot |
| P352 | H4 1-23 | Ac-SGRGKGGKGLGKGGAKRHRK(Ac)VLRD-Peg-Biot |
| P353 | H4 1-23 | Ac-pSGRGK(Ac)GGK(Ac)GLGK(Ac)GGAK(Ac)RHRKVLR-Peg-Biot |
| P354 | H4 1-23 | Ac-SGRGK(Ac)GGK(Ac)GLGK(Ac)GGAK(Ac)RHRK(Ac)VLR-Peg-Biot |
| P357 | H4 1-23 | Ac-pSGRGKGGKGLGKGGAKRHRKVLR-Peg-Biot |
| P358 | H4 1-23 | pSGRGKGGKGLGKGGAKRHRKVLR-Peg-Biot |
| P359 | H4 1-23 | Ac-SGRGK(Ac)GGK(Ac)GLGKGGAKRHRKVLR-Peg-Biot |
| P360 | H4 1-23 | Ac-SGRGK(Ac)GGKGLGKGGAK(Ac)RHRKVLR-Peg-Biot |
| P361 | H4 1-23 | Ac-SGRGK(Ac)GGKGLGKGGAKRHRK(Ac)VLR-Peg-Biot |
| P362 | H4 1-23 | Ac-SGRGKGGK(Ac)GLGK(Ac)GGAKRHRKVLR-Peg-Biot |
| P363 | H4 1-23 | Ac-SGRGKGGK(Ac)GLGKGGAKRHRK(Ac)VLR-Peg-Biot |
| P364 | H4 1-23 | Ac-SGRGKGGKGLGK(Ac)GGAK(Ac)RHRKVLR-Peg-Biot |
| P365 | H4 1-23 | Ac-SGRGKGGKGLGK(Ac)GGAKRHRK(Ac)VLR-Peg-Biot |
| P366 | H4 1-23 | Ac-SGRGKGGKGLGKGGAK(Ac)RHRK(Ac)VLR-Peg-Biot |
| P368 | H4-H3 | Ac-H4[1-23]5xAc-Peg-K(H3[1-20]-Peg)-Peg-Biot |
| P369 | H4-H3 | Ac-H4[1-23]5xAc-Peg-K(H3[1-20]K4(Me3)-Peg)-Peg-Biot |
| P370 | H4 1-23 | Ac-SGRGQGGQGLGK(Ac)GGAQRHRQVLR-Peg-Biot |
| P371 | H4 1-23 | Ac-SGRGK(Me)GGKGLGKGGAKRHRICVLR-Peg-Biot |
| P372 | H4 1-23 | Ac-SGRGKGGK(Me)GLGKGGAKRHRKVLR-Peg-Biot |
| P373 | H4 1-23 | Ac-SGRGKGGKGLGK(Me)GGAKRHRKVLR-Peg-Biot |
| P379 | H4 1-23 | Ac-SGRGK(Ac)GGKGLGK(Ac)GGAK(Ac)RHRKVLR-Peg-Biot |
| P380 | H4 1-23 | Ac-SGRGK(Ac)GGK(Ac)GLGKGGAK(Ac)RHRKVLR-Peg-Biot |
| P381 | H4 1-23 | Ac-SGRGK(Me)GGK(Ac)GLGK(Ac)GGAK(Ac)RHRKVLR-Peg-Biot |
| P382 | H4 1-23 | Ac-SGRGK(Ac)GGK(Me)GLGK(Ac)GGAK(Ac)RHRKVLR-Peg-Biot |
| P383 | H4 1-23 | Ac-SGRGK(Ac)GGK(Ac)GLGK(Me)GGAK(Ac)RHRKVLR-Peg-Biot |
| | | H2A PEPTIDES |
| P300 | H2A 1-17 | Ac-SGRGKQGGKARAKAKTR-Peg-Biot |
| P301 | H2A 1-17 | Ac-SGRGK(Ac)QGGK(Ac)ARAK(Ac)AK(Ac)TR-Peg-Biot |
| P302 | H2A 1-17 | Ac-SGRGK(Ac)QGGKARAKAKTR-Peg-Biot |
| P303 | H2A 1-17 | Ac-pSGRGK(Ac)QGGKARAKAKTR-Peg-Biot |
| P304 | H2A 1-17 | Ac-SGR(Me2a)GK(Ac)QGGKARAKAKTR-Peg-Biot |
| P305 | H2A 1-17 | Ac-pSGR(Me2a)GK(Ac)QGGKARAKAKTR-Peg-Biot |
| P306 | H2A 1-17 | Ac-SGCitGK(Ac)QGGKARAKAKTR-Peg-Biot |
| P307 | H2A 1-17 | Ac-pSGCitGK(Ac)QGGKARAKAKTR-Peg-Biot |
| P308 | H2A 1-17 | Ac-pSGRGK(Ac)QGGK(Ac)ARAK(Ac)AK(Ac)TR-Peg-Biot |
| P309 | H2A 1-17 | SGRGK(Ac)QGGK(Ac)ARAK(Ac)AK(Ac)TR-Peg-Biot |
| P310 | H2A 1-17 | pSGRGK(Ac)QGGK(Ac)ARAK(Ac)AK(Ac)TR-Peg-Biot |
| P586 | H2A10-25 | Ac-SAAKASQSRSAKAGLT-Peg-Biotin |
| P587 | H2A10-25 | Ac-ARAKAKTRSSRAGLQF-Peg-Biotin |
| P311 | H2A.X | Biot-Peg-G132KKATQAS139QEY142-OH |
| P312 | H2A.X | Biot-Peg-G132KKATQApS139QEY142-OH |
| P314 | H2A | Ac-SAAKASAAAAAKAGLT-Peg-Biot |
| P809 | H2A.X | SGRGKTGGKARAKAKSR-Peg-Biotin |
| P810 | H2A.X | SGRGK(Ac)TGGKARAKAKSR-Peg-Biotin |
| P811 | H2A.X | SGRGK(Ac)TGGK(Ac)ARAK(Ac)AK(Ac)SR-Peg-Biotin |
| P625 | H2A.X | Ac-SGRGKTGGKARAKAKSR-Peg-Biotin |
| P626 | H2A.X | Ac-SGRGK(Ac)TGGKARAKAKSR-Peg-Biotin |
| | | H2B PEPTIDES |
| P400 | H2B 1-24 | PEPAKSAPAPKKGSKKAVTKAQKK-Peg-Biot |
| P401 | H2B 1-24 | PEPAK(Me3)SAPAPICKGSKKAVTKAQKK-Peg-Biot |
| P402 | H2B 1-24 | PEPAK(Me2)SAPAPKKGSKKAVTKAQKK-Peg-Biot |
| P403 | H2B 1-24 | PEPAK(Me)SAPAPKKGSKKAVTKAQKK-Peg-Biot |
| P408 | H2B 1-24 | PEPAKSAPAPKK(Ac)GSKKAVTKAQKK-Peg-Biot |
| P409 | H2B 1-24 | PEPAKSAPAPKKGSK(Ac)KAVTKAQKK-Peg-Biot |
| P410 | H2B 1-24 | PEPAKSAPAPKKGSKK(Ac)AVTKAQKK-Peg-Biot |
| P411 | H2B 1-24 | PEPAKSAPAPKKGSKKAVTK(Ac)AQKK-Peg-Biot |
| P412 | H2B 1-24 | PEPAKSAPAPIU((Ac)GSK(Ac)K(Ac)AVTK(Ac)AQKK-Peg-Biot |

Exemplary synthetic histone peptides of the present invention. The exemplary synthetic histone peptides were derived from human and yeast histone sequences, and encompass peptides that cover the major modified forms of histones H3, H4, H2A, H2B, H2A.Z (Htzl), and yeast CENPA (Cnpl). Abbreviations of post-translational modifications are as follows: cetylation (Ac), lysine monomethylation (KMe1), lysine di-methylation (KMe2), lysine tri-methylation (KMe3), arginine mono-methylation (RMe1), arginine asymmetric di-methylation (RMe2a), arginine symmetric di-methylation (RMe2s), phosphoserine (pS), phosphotyrosine (pT), and Citrullination (Cit).

According to some embodiments of the present invention, the amino acid sequence of a synthetic histone peptide is modeled after a naturally occurring histone sequence and modified to include different and/or additional post-translational modifications that may exist and/or to provide different combinations of post-translational modifications within the peptide sequence. A synthetic histone peptide sequence can comprise combinations of two or more naturally occurring histone sequences from the same histone and/or a different histone, such as 2, 3, 4, or more histones. For example, a synthetic histone peptide sequence can comprise 5 to 20 amino acids from H3 and 5 to 20 amino acids from H4. Combinations of naturally occurring histone sequences can allow for the testing and/or determination of how modifications on different regions of a histone and/or on different histones can affect protein binding.

A synthetic histone peptide of the present invention can be characterized by one or more chemical and/or biological assays and/or techniques known to those of skill in the art. Characterization of a synthetic histone peptide of the present invention can used to determine and/or ensure quality of a peptide and/or to determine the specific chemical composition of a peptide. In some embodiments of the present invention, a synthetic histone peptide of the present invention is characterized using one or more of the following: high-performance liquid chromatography; mass spectrometry, such as electrospray mass spectrometry and matrix-assisted laser desorption mass spectrometry; nuclear magnetic resonance; or Edman degradation, including automated Edman degradation

A synthetic histone peptide of the present invention can have a purity of at least about 75% or more, such as about 80%, 85%, 90%, 95%, 99%, or more prior to being combined with another peptide and/or compound and/or used in an array of the present invention and/or method of the present invention. In particular embodiments of the present invention, a synthetic histone peptide of the present invention has a purity of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range therein.

One aspect of the present invention comprises, consists essentially of, or consists of a plurality of synthetic histone peptides of the present invention. A “plurality” as used herein refers to a group of two or more different peptides. In particular embodiments of the present invention, a plurality of synthetic histone peptide can comprise 5, 10, 15, 20, 25, 30, 35, 40, 45, 50, 55, 60, 65, 70, 75, 80, 85, 90, 95, 100, 125, 150, 175, 200, 250, 300, or more, or any range therein, different synthetic histone peptides. In some embodiments of the present invention, a plurality includes at least 5 synthetic histone peptides from Table 1 and/or Table 2.

Another aspect of the present invention includes one or more peptide arrays that comprise, consist essentially of, or consist of a plurality of synthetic peptides of the present invention. In some embodiments of the present invention, a peptide array comprises a substrate comprising a surface and a plurality of synthetic histone peptides immobilized on the substrate surface. “Peptide array” as used herein, refers to a series of peptides arranged in a two or three dimensional manner. The peptides can be arranged in a pattern and/or ordered manner, such as a spiral or grid pattern, and/or in an irregular manner. In particular embodiments of the present invention, the peptides are arranged in grids and/or rows and columns with areas containing no peptides located between adjacent peptides.

“Substrate” as used herein refers to any material onto which a synthetic histone peptide of the present invention can be arranged and/or immobilized. Exemplary substrates include, but are not limited to, wafers, slides, well plates, and membranes. The substrate can be porous or nonporous and/or rigid or semi-rigid. The substrate can comprise one or more materials such as, but not limited to, polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, and divinylbenzene styrene-based polymers), agarose (e.g., Sepharose™), dextran (e.g., Sephadex™), cellulosic polymers and other polysaccharides, silica and silica-based materials, glass (e.g., controlled pore glass) and functionalized glasses, ceramics, or any combination thereof. In particular embodiments of the present invention the substrate comprises glass, such as, but not limited to, a glass slide.

The substrate comprises a surface. In some embodiments of the present invention, the substrate comprises one or more surface coatings. Exemplary surface coatings include, but are not limited to, polymers such as aminosilane and poly-L-lysine, microporous polymers (e.g., cellulosic polymers such as nitrocellulose), microporous metallic compounds (e.g., microporous aluminum), antibody-binding proteins, bisphenol A polycarbonate, and one half of a binding pair, such as streptavidin. In particular embodiments of the present invention, the substrate surface is coated with one half of a binding pair. “Binding pair” as used herein refers to any molecule that is able to specifically bind to another molecule, such as, but are not limited to, streptavidin to biotin, avidin to biotin, a receptor to a ligand, and an antibody to an antigen. In particular embodiments of the present invention, the substrate surface is coated with streptavidin.

A plurality of synthetic histone peptides of the present invention can be immobilized on the substrate surface of a peptide array of the present invention. “Immobilize” and grammatical variants thereof as used herein refer to a synthetic histone peptide being attached or bound (e.g., covalently or non-covalently) to the substrate surface either directly or indirectly. In particular embodiments of the present invention, a synthetic histone peptide is immobilized onto the substrate surface using a binding pair. This can allow for the synthetic histone peptide of the present invention to comprise a greater freedom of rotation compared to being directly bound and/or immobilized onto the substrate surface. In some embodiments of the present invention, streptavidin is coated on the substrate surface and biotin is attached to a synthetic histone peptide of the present invention.

A plurality of synthetic histone peptides of the present invention can be immobilized onto the substrate surface of a peptide array of the present invention at a high density. “High density” as used herein refers to a peptide array comprising a density of at least about 1,000 peptides per square centimeter of the substrate surface of the array. In particular embodiments of the present invention, a peptide array has a density of at least about 1,500, 2,000, 2,500, 5,000, 10,000, 25,000, 50,000, 75,000, 100,000, or more peptides per square centimeter of the substrate surface of the array. In some embodiments of the present invention, a peptide array has a density of at least about 2,600 peptides per square centimeter of the substrate surface of the array.

Immobilization of a synthetic histone peptide can be accomplished by spotting a peptide onto the substrate surface, “Spotting” and grammatical variants thereof as used herein, refer to contacting, placing, dropping, dripping, and the like, the peptide onto one or more specific locations (i.e., spots of any size or shape) on the substrate surface to immobilize the peptide onto the substrate surface. In some embodiments of the present invention, a synthetic histone peptide of the present invention can be immobilized on a peptide array once (i.e., one spot) or as a series of two or more spots on an array, such as 2, 4, 6, 8, 12, or more spots on an array, or any range therein. When a synthetic histone peptide of the present invention is spotted two or more times on a peptide array, the spots can be sequential (e.g., adjacent to one another) and/or nonsequential (e.g., placed in a different order and/or nonadjacent to one another) on the peptide array. In particular embodiments of the present invention, a synthetic histone peptide of the present invention can be immobilized on a peptide array as a series of six spots, two different times on the peptide array. A spot of a synthetic histone peptide of the present invention on the substrate surface can comprise a synthetic histone peptide having the same or a different sequence. For example, a spot can comprise a single synthetic histone peptide of the present invention (i.e., the spot comprises synthetic histone peptides comprising the same amino acid sequence) or a spot can comprise a combination of synthetic histone peptides of the present invention (i.e., the spot comprises synthetic histone peptides comprising two or more different amino acid sequences).

A spot can be from about 25 mm to about 700 mm in diameter or any range therein, such as from about 50 mm to about 500 mm or about 150 mm to about 300 mm in diameter. In some embodiments of the present invention, a spot is about 200 mm in diameter. The spacing between adjacent spots on the substrate surface of an array can be from 50 mm to 1000 mm or any range therein, such as from about 100 mm to about 800 mm or about 200 mm to about 500 mm. In some embodiments of the present invention, a spot is spaced apart from a next adjacent spot by about 375 mm. The diameter of one or more spots on a peptide array of the present invention and spacing between the one or more spots on a peptide array of the present invention can be substantially constant (i.e., varying by less than 15%, such as less than 10%, 5%, etc.) or the diameter and/or spacing can vary. In some embodiments of the present invention, the diameter of one or more spots and/or the spacing between the one or more spots on a peptide array of the present invention can be manipulated and/or designed to accommodate one or more features desired for a peptide array of the present invention. For example, the diameter of one or more spots and/or spacing between the one or more spots on a peptide array of the present invention can be changed depending on the number of peptides desired to be immobilized on the peptide array.

As described above, the amino acid sequence of a synthetic histone peptide of the present invention can be similar to a N-terminal tail of a histone, a C-terminal tail of a histone, an internal region of a histone, or any combination thereof. A synthetic histone peptide of the present invention can be immobilized on a peptide array at either terminus (i.e., the N-terminus or C-terminus of the synthetic histone peptide) or at any location of the peptide (e.g., the middle of the peptide). In some embodiments of the present invention, the N-terminus or C-terminus of the synthetic histone peptide is immobilized on a peptide array. When a synthetic histone peptide comprises an amino acid sequence similar to a naturally occurring histone amino acid sequence in the N-terminal tail of a histone, then the C-terminus of the synthetic histone peptide is immobilized on the substrate surface. Similarly, when a synthetic histone peptide comprises an amino acid sequence similar to a naturally occurring histone amino acid sequence from the C-terminal tail of a histone, then the N-terminus of the synthetic histone peptide is immobilized on the substrate surface. This can allow for the synthetic histone peptide to better model a naturally occurring histone amino acid sequence.

A plurality of synthetic histone peptides of the present invention can comprise one or more of the features described above for a synthetic histone peptide of the present invention. In some embodiments of the present invention, one or more portions of the synthetic histone peptides on a peptide array comprise one or more features described above for a synthetic histone peptide of the present invention. The various portions of synthetic histone peptides may or may not overlap. A “portion” as used herein can refer to any fraction of the total number of synthetic histone peptides in a plurality or on a peptide array, such as about 1% to about 100% of the total number of synthetic histone peptides on a peptide array or any range therein, such as about 5% to about 95% or about 20% to about 50%. In particular embodiments of the present invention, a portion refers to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or any range therein. For example, a portion (e.g., about 50% or more) of the synthetic histone peptides can comprise at least one post-translational modification and/or a portion (e.g., about 50% or more) of the synthetic histone peptides can be at least 21 amino acids in length. These two portions may contain the same and/or different synthetic histone peptides. Thus, as those skilled in the art will appreciate, a plurality of synthetic histone peptides of the present invention can provide a large number of peptides with one or more features that can be the same and/or different from one another.

In some embodiments of the present invention, a peptide array of the present invention can further comprise a positive control. A positive control can aid in determining the quality of the spotting of a synthetic histone peptide of the present invention. A positive control can be bound to the substrate surface using a binding pair, such as, but not limited to, streptavidin and biotin. In particular embodiments of the present invention, a positive control is separate from a synthetic histone peptide of the present invention. Thus, in some embodiments of the present invention, a positive control does not bind, attach, and/or immobilize to the substrate surface using a synthetic histone peptide and/or is not bound and/or attached to the synthetic histone peptide.

A positive control of the present invention can comprise any compound that is detectable, such as, but not limited to, a fluorescent compound. In some embodiments of the present invention, a fluorescent compound is bound to one half of a binding pair, such as, but not limited to, biotin. Exemplary fluorescent compounds include, but are not limited to, fluoresceins, such as TET (Tetramethyl fluorescein), 2′,7′-dimethoxy-4′,5′-dichloro-6-carboxyfluorescein (JOE),6-carboxyfluorescein (HEX) and 5-carboxyfluorescein (5-FAM); phycoerythrins; resorufin dyes; coumarin dyes; rhodamine dyes, such as 6-carboxy-X-rhodamine (ROX); cyanine dyes; BODIPY dyes; quinolines; pyrenes; N,N,N′,N′-tetramethyl-6-carboxyrhodamine (TAMRA); acridine; stilbene; Texas Red; as well as derivatives thereof. In some embodiments of the present invention, the fluorophore is a rhodamine dye or a BODIPY dye and in other embodiments the fluorophore is 6-aminoquinoline. In particular embodiments of the present invention, the positive control is a fluorescein.

The present invention also encompasses methods of using a synthetic histone peptide of the present invention, a plurality of synthetic histone peptides of the present invention and/or a peptide array of the present invention. In certain embodiments of the present invention, a synthetic histone peptide of the present invention, a plurality of synthetic histone peptides of the present invention, and/or a peptide array of the present invention can be utilized in one or more chemical and/or biological assays, such as, but not limited to, protein assays, enzyme assays, antibody assays, cellular assays, or combinations thereof.

In some embodiments of the present invention, a method for determining the binding of a protein to a peptide is provided comprising providing a peptide array of the present invention, applying a protein to the peptide array, and detecting binding of the protein to one or more synthetic histone peptides in the peptide array.

In other embodiments of the present invention, a method for detecting the influence of neighboring post-translational modifications on protein binding is provided comprising providing a peptide array of the present invention, applying a protein to the peptide array, detecting binding of the protein to one or more synthetic histone peptides in the peptide array, and comparing the sequences of the synthetic histone peptides bound to the protein, thereby detecting the influence of neighboring post-translational modifications on protein binding. In some embodiments of the present invention, the method for detecting the influence of neighboring post-translational modifications on protein binding comprises providing a peptide array comprising a plurality of synthetic histone peptides of the present invention, wherein the plurality comprises peptides with a similar sequence (e.g., peptides modeled after a particular sequence from one or more histones). The plurality of synthetic histone peptides with a similar sequence can comprise peptides with no post-translational modifications, peptides with one post-translational modification, and peptides with more than one post-translational modifications. Thus, different combinations of post-translational modifications can be compared.

Binding of a protein to a peptide array of the present invention can be accomplished by methods known in the art. For example, protein binding can be detected by methods including, but not limited to, chemical and/or biological assays, such as, but not limited to, western blot methods, and/or techniques, such as, but not limited to, fluorescence, immunoprecipitation, and chromatography, or any combination thereof.

According to some embodiments of the present invention, a method of the present invention provides for the visual detection of protein binding to one or more synthetic histone peptides in a peptide array of the present invention. Any protein can be used in the methods of the present invention, such as but not limited to, an antibody, an enzyme, a histone-interacting protein, or any combination thereof. Exemplary antibodies include, but are not limited to, the antibodies listed in Table 3, below. Exemplary enzymes include, but are not limited to, peptidases, proteases, lipases, kinases, histone-modifying enzymes (e.g., methyltransferases, deacetylases, acetyltransferases, etc.), or any combination thereof. Exemplary histone-interacting proteins include, but are not limited to, CHD1, RAG2, BTPF, or any combination thereof.

The present invention is explained in greater detail in the following non-limiting Examples.

### EXAMPLES

### Example 1

Protein posttranslational modifications (PTMs), such as phosphorylation, methylation, acetylation, and ubiquitination, regulate many processes, such as protein degradation, protein trafficking, and mediation of protein-protein interactions [1]. Perhaps the best-studied PTMs are those found to be associated with histone proteins. More than 100 histone PTMs have been described, and they largely function by recruiting protein factors to chromatin, which in turn drives processes such as transcription, replication, and DNA repair [2]. Likewise, dozens of chromatin-associating factors have been identified that bind to distinct histone PTMs, and hundreds of modification specific histone antibodies have been developed to understand the in vivo function of these modifications [3, 4].

The enormous number of potential combinations of histone PTMs represents a major obstacle to our understanding of how PTMs regulate chromatin-templated processes, as well as to our ability to develop high-quality diagnostic tools for chromatin and epigenetic studies.

The same obstacle applies to other proteins regulated by combinatorial PTMs: for example, p53, RNA polymerase, and nuclear receptors [5-7]. To that end, we developed a peptide array-based platform to begin to address how both histone-interacting proteins and antibodies recognize combinations of PTMs. We focused primarily on the recognition of PTMs associated with the N-terminal tail of histone H3, but this approach is useful for the study of other histone modifications and combinatorial PTMs found on other nonhistone proteins.

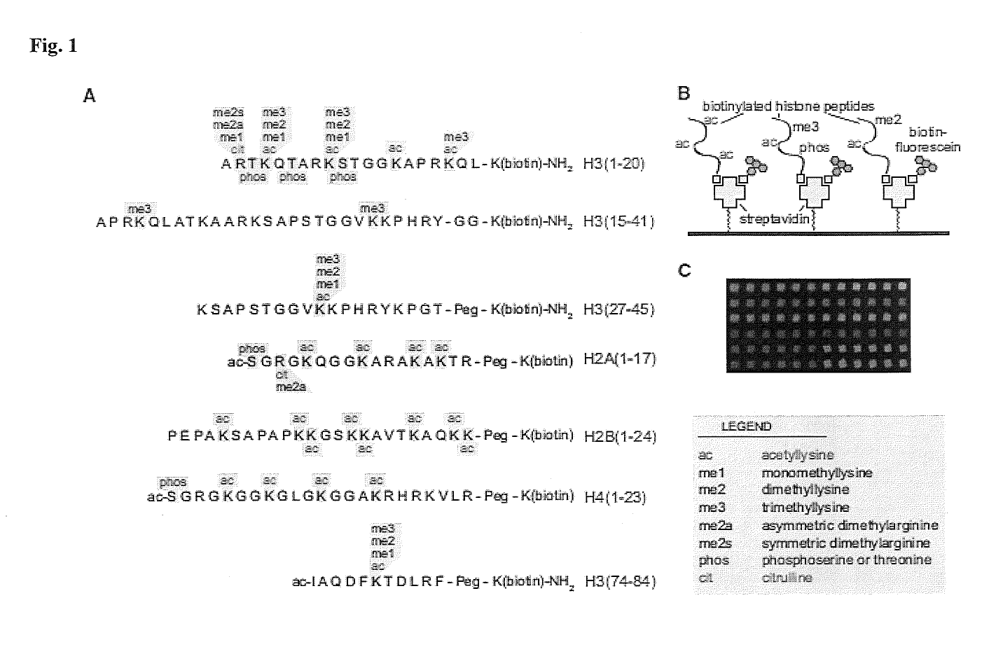

We generated a library of 110 synthetic histone peptides bearing either single or combinatorial PTMs and a biotin moiety for immobilization (FIG. 1 and Table 2). Prior to printing, all peptides were subjected to rigorous quality control to verify their accuracy. This is significant because extensive peptide purification and mass spectrometric analysis is not possible with other recently described array technologies used to study combinatorial histone PTMs [8]. Another significant advancement in our method was the introduction of a biotinylated fluorescent tracer molecule, which served as a positive control for the quality of our printing in all experiments. Lastly, peptides were printed as a series of six spots, two times per slide by two different pins, yielding 24 independent measurements of every binding interaction per slide. These measures were adopted to minimize binding artifacts due to pin variation or inconsistencies on the slide surface. Thus, these arrays offer a large number of extensively characterized histone peptide substrates suitable for the assessment of effector protein or antibody binding.

TABLE 2
List of peptide sequences and identifying number corresponding to the internal
tracking number. Omitted numbers code for peptides not used in this study.

| Peptide | |
|---|---|
| # | Sequence |
| | H3 [1-20] |
| 1 | ARTKQTARKSTGGKAPRKQL-K(Biot)-NH₂ |
| 2 | ARTKQTARKSTGGK(Ac)APRKQL-K(Biot)-NH₂ |
| 3 | ARTKQTARK(Ac)STGGKAPRKQL-K(Biot)-NH₂ |
| 4 | ARTK(Ac)QTARKSTGGKAPRKQL-K(Biot)-NH₂ |
| 5 | ARTK(Ac)QTARKSTGGK(Ac)APRKQL-K(Biot)-NH₂ |
| 6 | ARTKQTARK(Ac)STGGK(Ac)APRKQL-K(Biot)-NH₂ |
| 7 | ARTK(Ac)QTARK(Ac)STGGKAPRKQL-K(Biot)-NH₂ |
| 8 | ARTK(Ac)QTARK(Ac)STGGK(Ac)APRKQL-K(Biot)-NH₂ |
| 10 | ARTKQTARKSTGGKAPRK(Ac)QL-K(Biot)-NH₂ |
| 11 | ARTKQTARKSTGGK(Ac)APRK(Ac)QL-K(Biot)-NH₂ |
| 12 | ARTKQTARK(Ac)STGGKAPRK(Ac)QL-K(Biot)-NH₂ |
| 13 | ARTK(Ac)QTARKSTGGKAPRK(Ac)QL-K(Biot)-NH₂ |
| 14 | ARTKQTARK(Ac)STGGK(Ac)APRK(Ac)QL-K(Biot)-NH₂ |
| 15 | ARTK(Ac)QTARKSTGGK(Ac)APRK(Ac)QL-K(Biot)-NH₂ |
| 16 | ARTK(Ac)QTARK(Ac)STGGKAPRK(Ac)QL-K(Biot)-NH₂ |
| 17 | ARTK(Ac)QTARK(Ac)STGGK(Ac)APRK(Ac)QL-K(Biot)-NH₂ |
| 18 | ARTK(Me₃)QTARKSTGGKAPRKQL-K(Biot)-NH₂ |
| 19 | ARTK(Me₃)QTARK(Ac)STGGKAPRKQL-K(Biot)-NH₂ |
| 20 | ARTK(Me₃)QTARKSTGGK(Ac)APRKQL-K(Biot)-NH₂ |
| 21 | ARTK(Me₃)QTARKSTGGKAPRK(Ac)QL-K(Biot)-NH₂ |
| 22 | ARTK(Me₃)QTARK(Ac)STGGK(Ac)APRKQL-K(Biot)-NH₂ |
| 23 | ARTK(Me₃)QTARK(Ac)STGGKAPRK(Ac)QL-K(Biot)-NH₂ |
| 24 | ARTK(Me₃)QTARKSTGGK(Ac)APRK(Ac)QL-K(Biot)-NH₂ |
| 25 | ARTK(Me₃)QTARK(Ac)STGGK(Ac)APRK(Ac)QL-K(Biot)-NH₂ |
| 26 | ARpTK(Me₃)QTARK(Ac)STGGK(Ac)APRK(Ac)QL-K(Biot)-NH₂ |
| 27 | ARpTK(Me₃)QTARKSTGGKAPRKQL-K(Biot)-NH₂ |
| 28 | AR(Me₂a)pTK(Me₃)QTARK(Ac)STGGK(Ac)APRK(Ac)QL-K(Biot)-NH₂ |
| 29 | AR(Me₂a)pTK(Me₃)QTARKSTGGKAPRKQL-K(Biot)-NH₂ |
| 30 | AR(Me₂a)TK(Me₃)QTARKSTGGKAPRKQL-K(Biot)-NH₂ |
| 31 | 5-Fam-ARTKQTARKSTGGKAPRKQL-K(Biot)-NH₂ |
| 32 | ARTK(Me₂)QTARKSTGGKAPRKQL-K(Biot)-NH₂ |
| 33 | ARTK(Me₂)QTARK(Ac)STGGK(Ac)APRK(Ac)QL-K(Biot)-NH₂ |
| 34 | ARTK(Me)Q TARKSTGGKAPRKQL-K(Biot)-NH₂ |
| 35 | ARTK(Me)QTARK(Ac)STGGK(Ac)APRK(Ac)QL-K(Biot)-NH₂ |
| 36 | ARTKQTARKpSTGGKAPRKQL-K(Biot)-NH₂ |
| 37 | ARTK(Ac)QTARK(Ac)pSTGGK(Ac)APRK(Ac)QL-K(Biot)-NH₂ |
| 38 | ARTK(Me₃)QTARKpSTGGKAPRKQL-K(Biot)-NH₂ |
| 39 | ARTK(Me₃)QTARK(Ac)pSTGGK(Ac)APRK(Ac)QL-K(Biot)-NH₂ |
| 40 | AR(Me₂a)TK(Me₃)QTARKpSTGGKAPRKQL-K(Biot)-NH₂ |
| 41 | AR(Me2a)TK(Me₃)QTARK(Ac)pSTGGK(Ac)APRK(Ac)QL-K(Biot)-NH₂ |
| 42 | ARTKQTARK(Me₃)STGGKAPRKQL-K(Biot)-NH₂ |
| 43 | ARTK(Ac)QTARK(Me₃)STGGK(Ac)APRK(Ac)QL-K(Biot)-NH₂ |
| 44 | ARTK(Me₂)QTARK(Ac)STGGKAPRK(Ac)QL-K(Biot)-NH₂ |
| 45 | ARTK(Me)QTARK(Ac)STGGKAPRK(Ac)QL-K(Biot)-NH₂ |
| 47 | AR(Me₂a)TKQTARKSTGGKAPRKQL-K(Biot)-NH₂ |
| 48 | AR(Me₂a)TK(Ac)QTARK(Ac)STGGK(Ac)APRK(Ac)QL-K(Biot)-NH₂ |
| 50 | AR(Me₂a)TK(Me₃)QTARK(Ac)STGGK(Ac)APRK(Ac)QL-K(Biot)-NH₂ |
| 51 | AR(Me)TK(Me₃)QTARKSTGGKAPRKQL-K(Biot)-NH₂ |
| 52 | AR(Me)TK(Me₃)QTARK(Ac)STGGK(Ac)APRK(Ac)QL-K(Biot)-NH₂ |
| 53 | ACitTKQTARKSTGGKAPRKQL-K(Biot)-NH₂ |
| 54 | ACitTK(Me₃)QTARKSTGGKAPRKQL-K(Biot)-NH₂ |
| 55 | ACitTK(Me₃)QTARK(Ac)STGGK(Ac)APRK(Ac)QL-K(Biot)-NH₂ |
| 56 | ACitTK(Ac)QTARK(Ac)STGGK(Ac)APRK(Ac)QL-K(Biot)-NH₂ |
| | H4 [1-23] |
| 58 | Ac-SGRGKGGKGLGKGGAKRHRKVLR-Peg-Biot |
| 59 | Ac-SGRGK(Ac)GGK(Ac)GLGK(Ac)GGAK(Ac)RHRKVLR-Peg-Biot |
| 66 | Ac-SGRGK(Ac)GGKGLGKGGAKRHRKVLR-Peg-Biot |
| 67 | Ac-SGRGKGGK(Ac)GLGKGGAKRHRKVLR-Peg-Biot |
| 68 | Ac-SGRGKGGKGLGK(Ac)GGAKRHRKVLR-Peg-Biot |
| 69 | Ac-SGRGKGGKGLGKGGAK(Ac)RHRKVLR-Peg-Biot |
| 70 | Ac-SGRGK(Ac)GGKGLGK(Ac)GGAKRHRKVLR-Peg-Biot |
| 71 | Ac-SGRGKGGK(Ac)GLGKGGAK(Ac)RHRKVLR-Peg-Biot |
| 72 | Ac-SGRGK(Ac)GGK(Ac)GLGK(Ac)GGAKRHRKVLR-Peg-Biot |
| | H3 [15-41] |
| 90 | Ac-APRK¹⁸QLATK²³AARK²⁷SAPSTGGVK³⁶K³⁷PHRY-GG-K(Biot)-NH₂ |
| 91 | Ac-APRK(Me₃)QLATKAARKSAPSTGGVKKPHRY-GG-K(Biot)-NH₂ |
| 93 | Ac-APRKQLATKAARKSAPSTGGVK(Me₃)KPHRY-GG-K(Biot)-NH₂ |
| 95 | Ac-APRK(Me₃)QLATKAARKSAPSTGGVK(Me₃)KPHRY-GG-K(Biot)-NH₂ |
| | H3 [74-84] |
| 100 | Ac-IAQDFK⁷⁹TDLRF-Peg-K(Biot)-NH₂ |
| 101 | Ac-IAQDFK(Me₃)TDLRF-Peg-K(Biot)-NH₂ |
| 102 | Ac-IAQDFK(Me₂)TDLRF-Peg-K(Biot)-NH₂ |
| 103 | Ac-IAQDFK(Me)TDLRF-Peg-K(Biot)-NH₂ |
| 104 | IAQDFKTDLRF-Peg-K(Biot)-NH₂ |
| | H3 [27-45] |
| 120 | KSAPSTGGVK(Me₃)KPHRYKPGT-G-K(Biot)-NH₂ |
| 121 | KSAPSTGGVK(Me₂)KPHRYKPGT-G-K(Biot)-NH₂ |
| 122 | KSAPSTGGVK(Me)KPHRYKPGT-G-K(Biot)-NH₂ |
| 123 | KSAPSTGGVK(Ac)KPHRYKPGT-GG-K(Biot)-NH₂ |
| 124 | KSAPSTGGVK³⁶K³⁷PHRYKPGT-GG-K(Biot)-NH₂ |
| | H3 [1-20] |
| 132 | ARTK(Me₃)QTARK(Me₃)STGGKAPRKQL-K(Biot)-NH₂ |
| 133 | ARTKQTARK(Me₂)STGGKAPRKQL-K(Biot)-NH₂ |
| 134 | ARTKQTARK(Me)STGGKAPRKQL-K(Biot)-NH₂ |
| 137 | ARTKQTARKSTGGKAPRK(Me3)QL-K(Biot)-NH₂ |
| 138 | ARTKQTARKSTGGKAPRK(Me2)QL-K(Biot)-NH₂ |
| 139 | ARTKQTARKSTGGKAPRK(Me)QL-K(Biot)-NH₂ |
| 144 | ARTKQTARK(Ac)phSTGGKAPRKQL-K(Biot)-NH₂ |
| 145 | ARTKQTARK(Me₃)phSTGGKAPRKQL-K(Biot)-NH₂ |
| 146 | ARTKQTARK(Me₂)phSTGGKAPRKQL-K(Biot)-NH₂ |
| 147 | ARTKQTARK(Me)phSTGGKAPRKQL-K(Biot)-NH₂ |
| 148 | ARTK(Me₃)QTARK(Ac)phSTGGKAPRKQL-K(Biot)-NH₂ |
| 157 | AR(Me₂s)TK(Me₃)QTARKSTGGKAPRKQL-K(Biot)-NH₂ |
| 162 | ARTKQpTARKSTGGKAPRKQL-K(Biot)-NH₂ |
| 163 | ARTK(Me₃)QpTARKSTGGKAPRKQL-K(Biot)-NH₂ |
| 164 | ARTK(Me₂)QpTARKSTGGKAPRKQL-K(Biot)-NH₂ |
| 165 | ARTKQpTARK(Ac)STGGK(Ac)APRK(Ac)QL-K(Biot)-NH₂ |
| 166 | ARTK(Me3)QpTARK(Ac)STGGK(Ac)APRK(Ac)QL-K(Biot)-NH₂ |
| 167 | ARTK(Me2)QpTARK(Ac)STGGK(Ac)APRK(Ac)QL-K(Biot)-NH₂ |
| | H2A[1-17] |
| 300 | Ac-SGRGK⁵QGGK⁹ARAK¹³AK¹⁵TR-Peg-Biot |
| 301 | Ac-SGRGK(Ac)QGGK(Ac)ARAK(Ac)AK(Ac)TR-Peg-Biot |
| 302 | Ac-SGRGK(Ac)QGGKARAKAKTR-Peg-Biot |
| 303 | Ac-pSGRGK(Ac)QGGKARAKAKTR-Peg-Biot |
| 304 | Ac-SGR(Me₂a)GK(Ac)QGGKARAKAKTR-Peg-Biot |
| 305 | Ac-pSGR(Me₂a)GK(Ac)QGGKARAKAKTR-Peg-Biot |
| 306 | Ac-SCitGK(Ac)QGGKARAKAKTR-Peg-Biot |
| 307 | Ac-pSGCitGK(Ac)QGGKARAKAKTR-Peg-Biot |
| 308 | Ac-pSGRGK(Ac)QGGK(Ac)ARAK(Ac)AK(Ac)TR-Peg-Biot |
| 309 | SGRGK(Ac)QGGK(Ac)ARAK(Ac)AK(Ac)TR-Peg-Biot |
| 310 | pSGRGK(Ac)QGGK(Ac)ARAK(Ac)AK(Ac)TR-Peg-Biot |
| | H2B[1-24] |
| 400 | PEPAKSAPAPKKGSKKAVTKAQKK-Peg-Biot |
| 401 | PEPAK(Me₃)SAPAPKKGSKKAVTKAQKK-Peg-Biot |
| 402 | PEPAK(Me₂)SAPAPKKGSKKAVTKAQKK-Peg-Biot |
| 403 | PEPAK(Me)SAPAPKKGSKKAVTKAQKK-Peg-Biot |

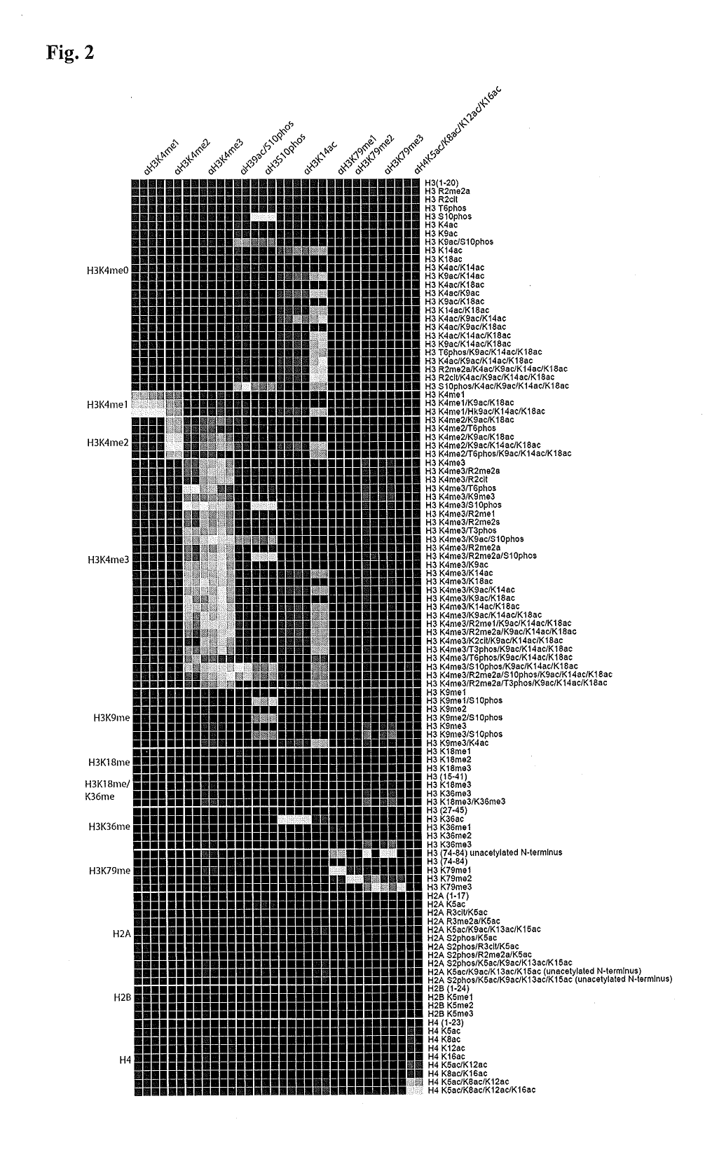

We initially used our arrays to ask two fundamental questions regarding the recognition of histone PTMs: (1) How well do modification-directed antibodies recognize their intended epitope? and (2) What impact, if any, do combinatorial PTMs have on antibody recognition? We tested more than 20 commercially available antibodies raised against individual modifications on histone tails (see Tables 3 and 4) for information regarding antibodies and experimental conditions). Generally, we found that antibodies were reasonably proficient at recognizing their target modification (FIG. 2). However, we found several exceptions, notably the discrimination between different methyllysine states by methyl-specific antibodies and the recognition of histone H3 lysine 14 acetylation (H3K14ac).

TABLE 3
List of antibodies and sources.

| Modification | Antibody | Source | Supplier | Catalog | Lot |
|---|---|---|---|---|---|
| H3K4me1 | polyclonal | rabbit | upstate | 07-436 | 30218 |
| | polyclonal | rabbit | millipore | 07-436 | DAM1687548 |
| H3K4me2 | polyclonal | rabbit | active motif | 39142 | 168 |
| H3K4me3 | polyclonal | rabbit | active motif | 39160 | 1609004 |
| | polyclonal | rabbit | millipore | 07-473 | DAM1623866 |
| | monoclonal | mouse | abcam | ab1012 | 761207 |
| H3K14ac | polyclonal | rabbit | active motif | 39616 | 11709001 |
| | polyclonal | rabbit | millipore | 07-353 | DAM1548623 |
| | polyclonal | rabbit | abcam | ab46984 | 730270 |
| H3S10phos | polyclonal | rabbit | active motif | 39253 | 8308001 |
| H3K9acS10phos | polyclonal | rabbit | cell signaling | 9711 | ref 10/2008 |
| H3K79me1 | polyclonal | rabbit | active motif | 39146 | 172 |
| H3K79me2 | monoclonal | rabbit | milipore | 04-835 | DAM1527889 |
| H3K79me3 | polyclonal | rabbit | abcam | ab2621 | 809870 |
| | polyclonal | rabbit | abcam | ab2621 | 441039 |
| H4tetraacetyl | polyclonal | rabbit | active motif | 39179 | 1008001 |

TABLE 4
Reaction conditions for all antibodies tested.

| Barcode | Antibody | Dilution |
|---|---|---|
| 343501 | H3K79me3 abcam new lot | 1:1000 |
| 343502 | H3K4me1 upstate | 1:1000 |
| 343503 | H4 tetraacetyl | 1:2000 |
| 343504 | H3K79me2 | 1:1000 |
| 343505 | H3K79me1 | 1:1000 |
| 343506 | H3K4me2 | 1:5000 |
| 343507 | H3K14Ac millipore | 1:5000 |
| 343508 | H3S10phos | 1:5000 |
| 343509 | H3K79me3 old lot | 1:3000 |
| 343510 | H3K79me2 | 1:1000 |
| 343511 | H3K4me3 active motif | 1:1000 |
| 343512 | H3K14Ac abcam | 1:1000 |
| 343513 | H3K14Ac active motif | 1:1000 |
| 343514 | H3K14Ac active motif | 1:1000 |
| 343515 | H3K14Ac millipore | 1:5000 |
| 343516 | H3K4me3 active motif | 1:1000 |
| 343517 | H3K4me3 millipore | 1:10000 |
| 343518 | H3K4me3 abcam | 2.5 μg/mL |
| 343520 | H3K4me2 | 1:5000 |
| 343521 | H3K14Ac abcam | 1:1000 |
| 343522 | H3K79me1 | 1:1000 |
| 343523 | H3k79me3 new | 1:3000 |
| 343524 | H3K4me3 Millipore | 1:10000 |
| 343525 | H3K9acS10phos | 1:1000 |
| 343544 | H4 tetraacetyl | 1:2000 |
| 343545 | H3K79me3 | 1:1000 |
| 343546 | H3K4me1 upstate | 1:1000 |
| 343547 | H3K4me3 abcam | 2.5 μg/mL |
| 343548 | H3S10phos | 1:5000 |
| 343549 | H3K4me1 millipore | 1:1000 |
| 343550 | H3K9acS10phos | 1:1000 |
| 343602 | H3K79me3 old lot | 1:3000 |
| 343604 | H3K4me1 millipore | 1:1000 |
| 343606 | H3S10phos | 1:5000 |
| 343614 | H3k79me3 abcam new lot | 1:3000 |

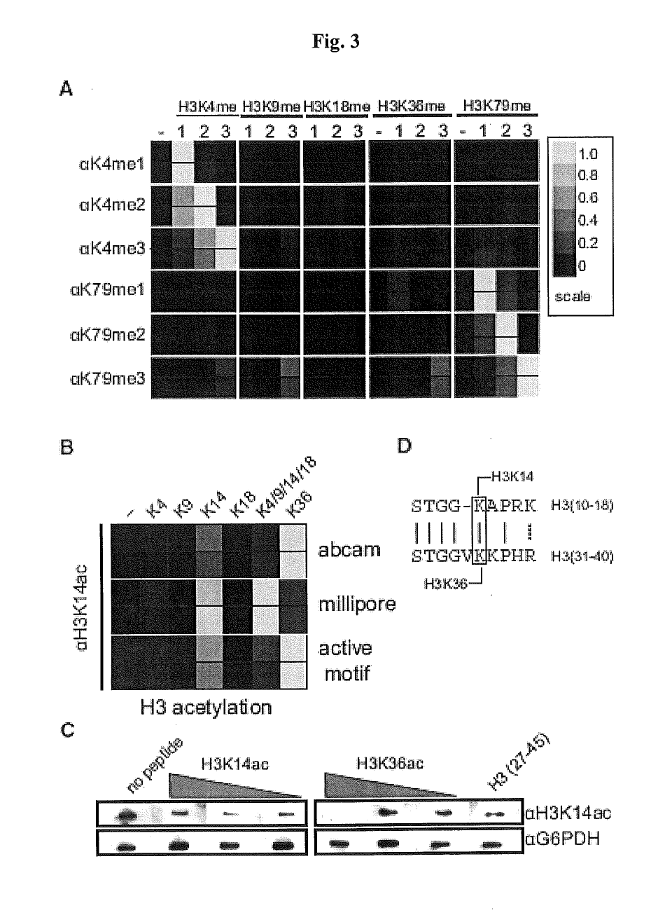

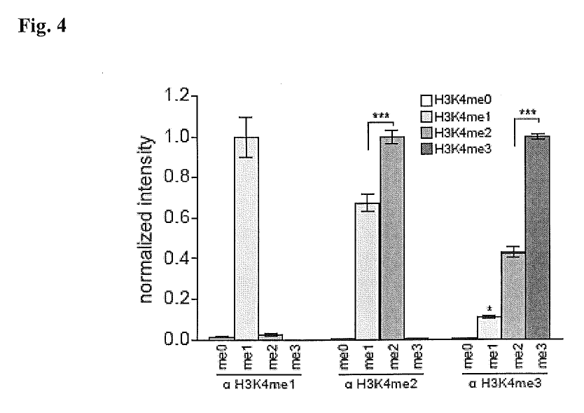

To explore methyllysine recognition, we tested the specificity of commercial antibodies raised against the three different methylated forms (mono-, di-, and trimethyl) of H3 at lysines 4 and 79 (H3K4me and H3K79me) (FIG. 3). These antibodies were generally specific for their target lysine residue; however, both the trimethyl- and dimethyl-directed antibodies showed measurable cross-reactivity with dimethyllysine and monomethyllysine, respectively (FIG. 3A and FIG. 4). This finding has particular biological importance, because each methylation state of a given histone lysine residue is thought to mediate different biological outcomes through the recruitment of distinct chromatin-associated factors [9]. For example, H3K4me3 is well correlated with transcriptional activation through the recruitment of histone acetyltransferases and the preinitiation complex of transcription [10-12]. Conversely, H3K4me2 was reported to recruit the Set3 histone deacetylase complex [9]. The ability to distinguish between these methyl states is therefore necessary to dissect how H3K4 methylation controls the balance of histone acetylation and/or deacetylation at transcribed genes.

We also tested a number of antibodies raised against acetyllysine found at position 14 of histone H3 (H3K14ac). Unlike lysine methylation, our arrays detected that several of these antibodies had difficulty in recognizing their target sequence, preferring acetylation at lysine 36 (H3K36ac) instead (FIG. 3B). Additionally, peptide competition assays verified the interaction between the H3K14 antibodies and the H3K36ac peptide (FIG. 3C). This result is likely explained by the fact that H3K14 and H31K36 are found in very similar sequence contexts and are acetylated by the same enzyme in vivo (FIG. 3D). Acetylation of both H3K14 and H3K36 is catalyzed by the histone acetyltransferase Gcn5 [13]. However, H3K14ac is reported to be recognized by the RSC complex in yeast, whereas H3K36ac has been reported to be recognized by the bromodomain of PCAF in human cells [14, 15]. Thus, misdetection of H3K36ac using H3K14ac-directed antibodies by either western blot or chromatin immunoprecipitation may obscure our understanding of chromatin-templated processes regulated by H31K14 acetylation.

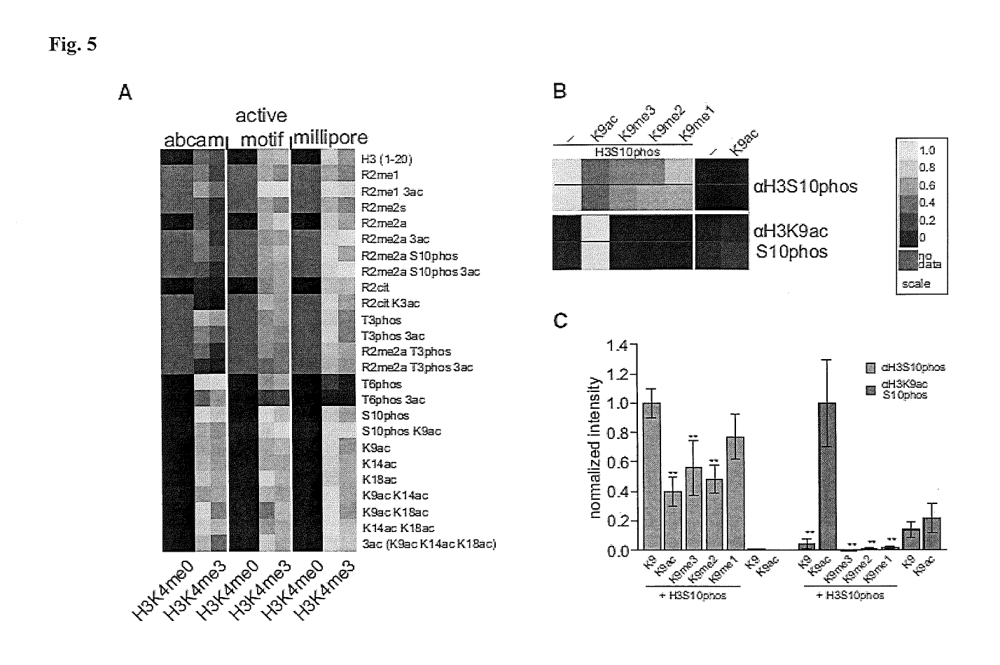

The large number of synthetic peptides containing combinatorial PTMs allowed us to additionally ascertain how PTM recognition is influenced by neighboring modifications. We therefore did further analysis of the H3K4me3 antibodies to determine how adjacent modifications affect substrate recognition. We observed that a monoclonal antibody widely used against H3K4me3 (Abeam; catalog number ab1012) is perturbed mainly by modification at histone H3 arginine 2 (H3R2) (FIG. 5A). In contrast, a widely used polyclonal antibody from Millipore (catalog number 07-473) was negatively influenced by H3T6 phosphorylation, and a similar antibody from Active Motif (catalog number 39160) was not particularly sensitive to any neighboring modifications (FIG. 5A).

We also examined the well-characterized PTM “switch” region on histone H3, where H3K9 is modified by either acetylation or methylation and where the neighboring serine 10 (H3S10) is a target for phosphorylation [16]. A polyclonal antibody (Active Motif; catalog number 39253) raised against H3S10 phosphorylation showed a statistically significant reduction in binding to peptides also modified at H3K9 (FIGS. 5B and 5C). In contrast, an antibody raised against both H3S10phos and H3K9ac (Cell Signaling; catalog number 9711) showed nearly absolute specificity for the peptide containing both modifications (FIGS. 5B and 5C). These data can be interpreted to suggest that biological changes in acetylation and methylation at H3K9 would influence the ability of antibodies derived against H3 S 10 phosphorylation to appropriately detect this mark. Such findings are significant, because H3S10 phosphorylation levels have already been found to change during the cell cycle and in response to histone deacetylase inhibitors [17-19].

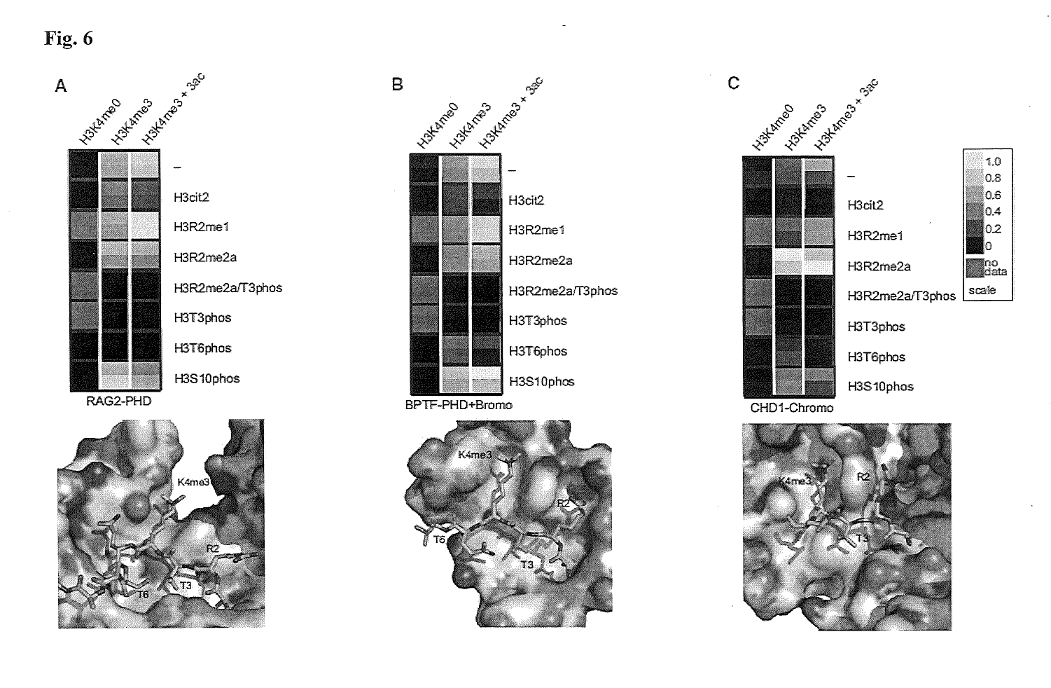

Collectively, our analysis of histone PTM-specific antibodies enabled us to uncover recognition of related (but off-target) sequences in addition to adjacent PTM effects. This finding is significant because several major ongoing initiatives aimed at mapping and understanding how histone PTMs regulate biology, such as the National Institutes of Health (NIH) Epigenomic Roadmap and ENCODE, heavily rely on modification-specific antibodies [20]. In addition to being a powerful diagnostic tool for the characterization of PTM-derived antibodies, we used our peptide array technology to measure how PTM codes affect the interaction of chromatin-associated proteins. Accordingly, we measured the binding of several domains known to interact with H3K4me3. We found that the PHD domain from the V(D)J recombination factor RAG2 was specific for H3K4me3 and was blocked by phosphorylation at either H3T3 or H3T6 (FIG. 6A). From the structure of the RAG2 PHD domain bound to H3K4me3 peptide [21], it can clearly be seen how H3T3 phosphorylation may disrupt binding. Varier and coworkers very recently published that H3T3 phosphorylation acts as a switch to control the binding of TAF3 PHD domain [22]. Thus, this may be a general mechanism for controlling gene expression during mitosis (when H3T3 is phosphorylated). Similarly, Garske and coworkers found that H3T6 phosphorylation may disrupt RAG2 binding [23].

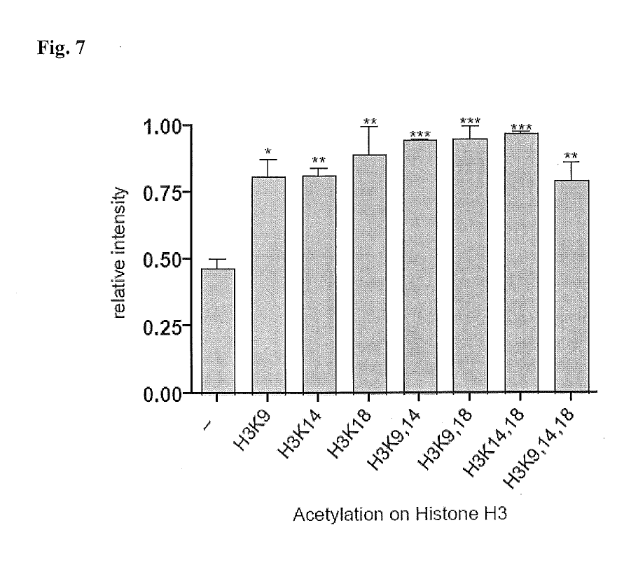

We next examined the tandem bromo-PHD domains of BPTF (subunit of the NURF ATP-dependent remodeling complex [24]). Our studies showed that the tandem domain was specific for H3K4me3 and also showed reduced binding in the presence of either H3T3 or H3T6 phosphorylation (FIG. 6B). However, both RAG2 and BPTF are blocked by citrulline, but not by methylation at position 2, suggesting a role for the positive charge of H3R2 in PHD domain binding. Notably, converting H3R2 to citrulline results in a loss of cationic charge and likely loss of ionic and hydrogen bonding interactions within the pockets of the two PHD domains (FIGS. 6A and 6B). Interestingly, our ability to synthesize and print long peptides (R20 amino acids) allowed us to observe greater interactions of BPTF (PHD-bromo) with H3K4me3 peptides also harboring acetylation. We found that multiple acetylations on H3 enhanced the binding of BPTF to H3K4me3 (FIG. 6B and FIG. 7), suggesting coordination between the methylbinding PHD domain and the acetyl-binding bromodomain to recognize multiple modifications on the histone H3 tail.

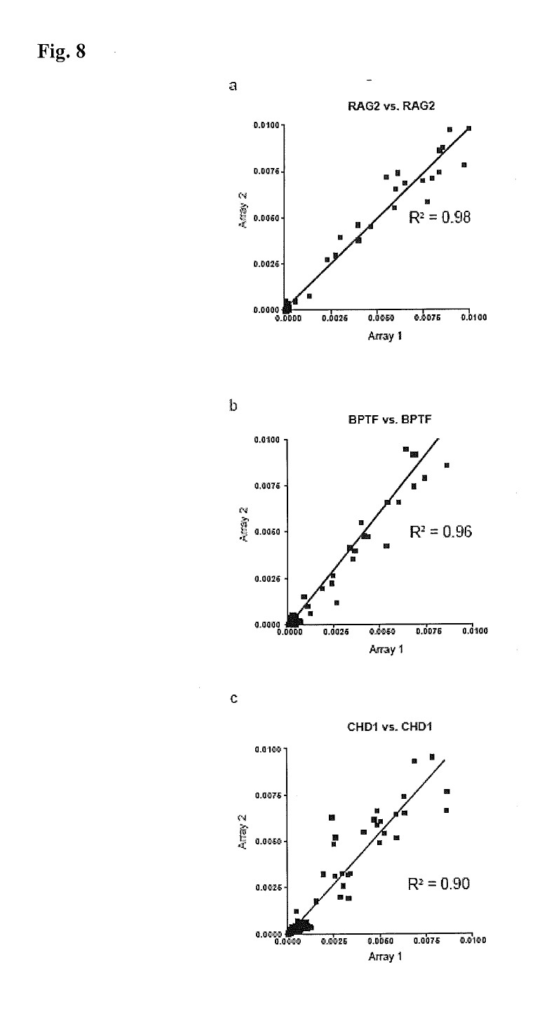

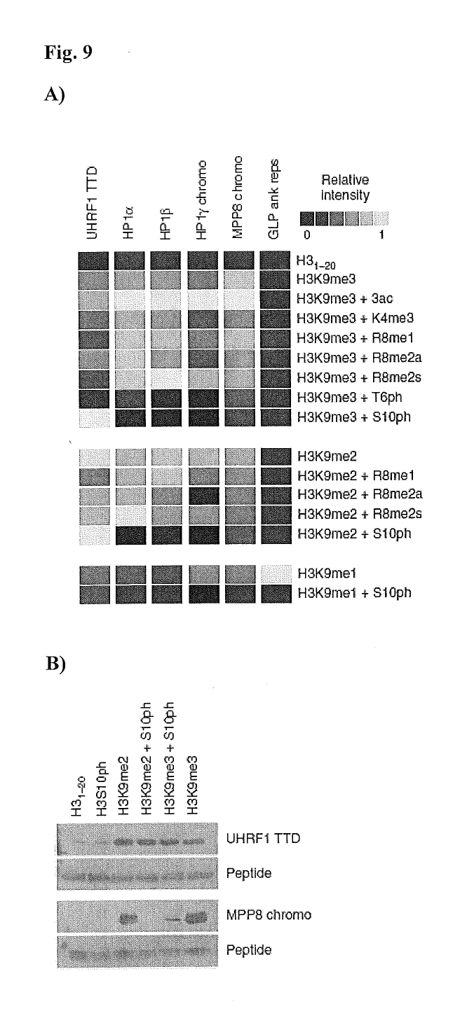

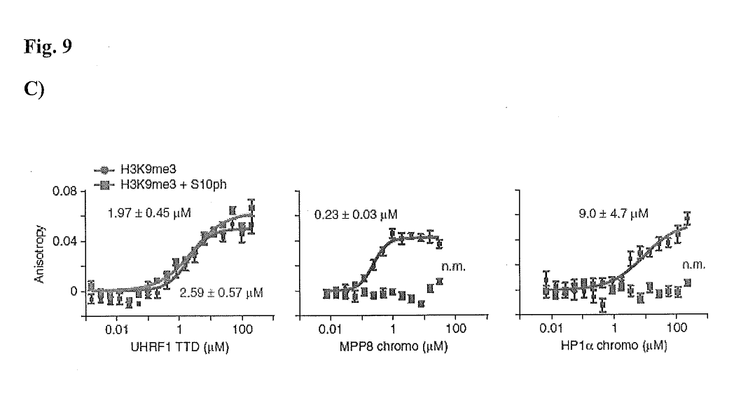

The chromodomain of human CHD1 is also known to recognize H3K4me3 but has a structurally distinct binding pocket from the PHD domains. We found that CHD1, like RAG2 and BPTF, preferentially bindsH3K4me3 and is also negatively influenced by phosphorylation at H3T3 and H3T6 (FIG. 6C). Interestingly, we also found that methylation of H3R2 appears to slightly enhance binding of CHD1, whereas citrullination at position 2 blocks this binding. Although the finding that H3R2 methylation reduces binding affinity of human CHD1 to H3K4me3 is in opposition to a previous report [25], this discrepancy may be due to the fact that Flanagan and coworkers used peptides labeled at the N terminus with fluorescein in their binding studies, which may have contributed to the binding. Consistent with our CHD1 findings, we and others have found that H3R2 methylation does not decrease CHD1 binding to H3K4me3 by either isothermal titration calorimetry (data not shown) or fluorescence polarization using C-terminally labeled peptides (Marcey Waters, personal communication). H3R2 methylation and H3K4me3 have been found to be mutually exclusive in yeast and humans [26, 27]. Thus, H3R2 methylation and H3K4me3 may function in specific circumstances to prevent the binding of effector proteins that promote gene transcription while facilitating the recruitment of CHD1(and possibly other factors) to genes in order to promote gene silencing. A comparison of the two arrays for RAG2, BPTF, and CHD1 was performed as shown in FIG. 8.

In conclusion, the complex patterns of histone PTMs are critical determinants of chromatin structure and function, but they also represent a significant challenge for future study. Although many protein domains that bind selectively to particular PTMs have been identified, little is known regarding how neighboring modifications inhibit or contribute to these interactions. Of equal importance is our understanding of how patterns of PTMs influence antibody recognition. In this case, detection of biologically important events could be blocked or misrepresented if neighboring modifications interfere with epitope recognition. Thus, our work underscores a need for more rigorous testing and characterization of histone-specific antibodies. Similar antibody concerns have been recently highlighted by other groups [20, 28]. The data sets for the antibodies and proteins described here, plus numerous additional antibodies, are available in FIG. 2 and from our website (http://www.med.unc.edu/wbstrahl/Arrays/index.htm). In addition, we will continue to characterize histone antibody specificities and post the data to our website as an ongoing resource for the chromatin community.

Finally, although several other peptide array approaches have been used to measure binding to histone PTMs [8, 29-31], our arrays and assay approaches offer several advantages. First, our array displays a large number of peptides carrying multiple PTMs that are fully characterized by high performance liquid chromatography (HPLC) and mass spectrometry (MS). Second, we take advantage of a biotin tracer molecule to provide an assessment of printing efficiency. Lastly, the high density of spotting allows us to perform statistical analysis of binding interactions. Although Liu et al. recently reported a similarly semiquantitative approach, their arrays were largely limited to peptides containing single PTMs, and the peptides were labeled via their N terminus, which could potentially occlude proteins and antibodies from recognizing modifications such as H3K4 methylation [30]. Furthermore, cellulose SPOT synthesis technology is limited by the inability to analytically characterize peptides [28]. In addition, a very elegant bead-based approach has been used to generate even larger peptide libraries and successfully characterize the binding of several protein factors to combinatorial histone PTMs [23]. However, our approach offers advantages in that we obtain binding data for each individual peptide and do not require sophisticated MS for the analysis.

### Experimental Procedures

### Antibodies

All primary antibodies tested are commercially available and are listed in Table 3. Secondary antibodies were Alexa Fluor 647 conjugated goat anti-rabbit IgG (catalog number A21244) and Alexa Fluor 647 conjugated rabbit anti-mouse IgG (catalog number A21239) antibodies from Invitrogen.

### Peptide Synthesis

All reagents were obtained from commercial suppliers (AnaSpec, EMD, and Apptec). The peptides, biotinylated at their C termini, were synthesized on either NovaPEG Rink amide resin (histone H3 peptides) or Biotin-PEG NovaTag resin (histone H2A, H2B, and H4 peptides) using fluorenylmethyloxycarbonyl (Fmoc) chemistry on a PS-3 automated peptide synthesizer (see Table 2 for the complete list of peptides). All standard amino acids were coupled using HATU and N-methylmorpholine in dimethylformamide (DMF). Fmoc deprotection was performed using 20% piperidine in DMF. Modified amino acid residues were coupled using HATU, HOAt, and N,N,-diisopropyletylamine in NMP, and the coupling of these residues was monitored using ninhydrin test and repeated when needed. Peptides were cleaved from the resins using a 2,5% TIS and 2.5% water in trifluoroacetic acid (TFA). After TFA evaporation and washing with diethyl ether, the peptides were lyophilized from an acetonitrile/water solution and purified via preparative HPLC using water-acetonitrile gradient (0.1% TFA in both solvents) on a Waters SymmetryShield RP-18 5 mm 19 3 150 mm column. All peptides were analyzed using matrix-assisted laser desorption/ionization time-of-flight mass spectrometry and analytical HPLC. The average purity of peptides was over 90% (analytical HPLC). Analytical data for all peptides mentioned in this paper is available on our website.

### Array Fabrication

Biotinylated peptides (25 mM final concentration) in printing buffer (10 mg/ml bovine serum albumin [BSA, Amresco], 0.3% Tween-20, and 10 mM biotinconjugated fluorescein added to 13 ArrayIt protein printing buffer) were arrayed onto SuperStreptavidin-coated slides (ArrayIt) using SMP6 stealth pins (˜200 mm spot diameter) and were arrayed onto OmniGrid100 arrayer (Digilab/Genomic Solutions) at ambient temperature and humidity (50%-60%) using the following printing parameters. To minimize effects from individual pins or localized imperfections in the substrate arrays, we arrayed samples as a series of six spots, two times on each slide at a spacing of 375 mm, as indicated in Table 5, and each peptide was printed by two different pins on each slide. After printing, slides were incubated overnight at 4° C. in a humidified environment to facilitate interaction between the biotinylated peptide and the streptavidin surface. Slides were then blocked for 1 hr at 4° C. with biotin-blocking buffer (Arrayft), washed three times with phosphate-buffered saline (PBS), dried with air, stored at 4° C., and used within 60 days.

TABLE 5
Map of peptide location on arrays - listed by identifying number. Each peptide was printed as a series
of 6 sequential spots on each of four subarrays. The layout of subarrays 1 and 3, and 2 and 4 are identical.
Parentheses denote the location within a subarray where a given peptide was printed.
subarray 1 and 3

| | | | | | | | |
|---|---|---|---|---|---|---|---|
| IgG | 1 | IgG | 2 | IgG | 3 | IgG | 4 |
| (A1-A6) | (A7-A12) | (A13-A18) | (A19-A24) | (A25-A30) | (A31-A36) | (A37-A42) | (A43-A48) |
| F | 5 | 100 | 6 | 120 | 7 | 58 | 8 |
| (B1-B6) | (B7-B12) | (B13-B18) | (B19-B24) | (B25-B30) | (B31-A36) | (B37-B42) | (B43-B48) |
| 90 | 10 | F | 11 | 121 | 12 | 59 | 13 |
| (C1-C6) | (C7-C12) | (C13-C18) | (C19-C24) | (C25-C30) | (C31-C36) | (C37-C42) | (C43-C48) |
| 91 | 14 | 101 | 15 | F | 16 | 66 | 17 |
| (D1-D6) | (D7-D12) | (D13-D18) | (D19-D24) | (D25-D30) | (D31-D36) | (D37-D42) | (D43-D48) |
| 93 | 18 | 102 | 19 | 122 | 20 | F | 21 |
| (E1-E6) | (E7-E12) | (E13-E18) | (E19-E24) | (E25-E30) | (E31-E36) | (E37-E42) | (E43-E48) |
| 95 | 22 | 103 | 23 | 123 | 24 | 67 | 25 |
| (F1-F6) | (F7-F12) | (F13-F18) | (F19-F24) | (F25-F30) | (F31-F36) | (F37-F42) | (F43-F48) |
| 69 | 26 | 104 | 27 | 123 | 28 | 68 | 29 |
| (G1-G6) | (G7-G12) | (G13-G18) | (G19-G24) | (G25-G30) | (G31-G36) | (G37-G42) | (G43-G48) |
| 162 | 145 | 144 | 137 | 147 | 138 | 148 | 139 |
| (H1-H6) | (H7-H12) | (H13-H18) | (H19-H24) | (H25-H30) | (H31-H36) | (H37-H42) | (H43-H48) |
| blank | blank | blank | blank | blank | blank | blank | blank |
| (I1-I6) | (I7-I12) | (I13-I18) | (I19-I24) | (I25-I30) | (I31-I36) | (I37-I42) | (I43-I48) |
| blank | blank | blank | blank | blank | blank | blank | blank |
| (J1-J6) | (J7-J12) | (J13-J18) | (J19-J24) | (J25-J30) | (J31-J36) | (J37-J42) | (J43-J48) |
| blank | blank | blank | blank | blank | blank | blank | blank |
| (K1-K6) | (K7-K12) | (K13-K18) | (K19-K24) | (K25-K30) | (K31-K36) | (K37-K42) | (K43-K48) |
| blank | blank | blank | blank | blank | blank | blank | blank |
| (L1-L6) | (L7-L12) | (L13-L18) | (L19-L24) | (L25-L30) | (L31-L36) | (L37-L42) | (L43-L48) |
| IgG | 30 | IgG | 32 | IgG | 33 | IgG | 34 |
| (M1-M6) | (M7-M12) | (M13-M18) | (M19-M24) | (M25-M30) | (M31-M36) | (M37-M42) | (M43-M48) |
| 70 | 35 | 301 | 36 | 305 | 37 | F | 38 |
| (N1-N6) | (N7-N12) | (N13-N18) | (N19-N24) | (N25-N30) | (N31-N36) | (N37-N42) | (N43-N48) |
| 71 | 39 | 302 | 40 | F | 41 | 309 | 42 |
| (O1-O6) | (O7-O12) | (O13-O18) | (O19-O24) | (O25-O30) | (O31-O36) | (O37-O42) | (O43-O48) |
| 72 | 43 | F | 44 | 306 | 45 | 310 | 157 |
| (P1-P6) | (P7-P12) | (P13-P18) | (P19-P24) | (P25-P30) | (P31-P36) | (P37-P42) | (P43-P48) |
| F | 47 | 303 | 48 | 307 | 50 | 400 | 51 |
| (Q1-Q6) | (Q7-Q12) | (Q13-Q18) | (Q19-Q24) | (Q25-Q30) | (Q31-Q36) | (Q37-Q42) | (Q43-Q48) |
| 300 | 52 | 304 | 53 | 308 | 54 | 401 | 55 |
| (R1-R6) | (R7-R12) | (R13-R18) | (R19-R24) | (R25-R30) | (R31-R36) | (R37-R42) | (R43-R48) |
| 402 | IgG | 403 | 167 | 56 | IgG | F | IgG |
| (S1-S6) | (S7-S12) | (S13-S18) | (S19-S24) | (S25-S30) | (S31-S36) | (S37-S42) | (S43-S48) |
| 163 | 146 | 164 | 132 | 165 | 133 | 166 | 134 |
| (T1-T6) | (T7-T12) | (T13-T18) | (T19-T24) | (T25-T30) | (T31-T36) | (T37-T42) | (T43-T48) |
| blank | blank | blank | blank | blank | blank | blank | blank |
| (U1-U6) | (U7-U12) | (U13-U18) | (U19-U24) | (U25-U30) | (U31-U36) | (U37-U42) | (U43-U48) |
| blank | blank | blank | blank | blank | blank | blank | blank |
| (V1-V6) | (V7-V12) | (V13-V18) | (V19-V24) | (V25-V30) | (V31-V36) | (V37-V42) | (V43-V48) |
| blank | blank | blank | blank | blank | blank | blank | blank |
| (W1-W6) | (W7-W12) | (W13-W18) | (W19-W24) | (W25-W30) | (W31-W36) | (W37-W42) | (W43-W48) |
| blank | blank | blank | blank | blank | blank | blank | blank |
| (X1-X6) | (X7-X12) | (X13-X18) | (X19-X24) | (X25-X30) | (X31-X36) | (X37-X42) | (X43-X48) |

subarray 2 and 4

| | | | | | | | |
|---|---|---|---|---|---|---|---|
| 134 | 166 | 133 | 165 | 132 | 164 | 146 | 163 |
| (A1-A6) | (A7-A12) | (A13-A18) | (A19-A24) | (A25-A30) | (A31-A36) | (A37-A42) | (A43-A48) |
| IgG | F | IgG | 56 | 167 | 403 | IgG | 402 |
| (B1-B6) | (B7-B12) | (B13-B18) | (B19-B24) | (B25-B30) | (B31-A36) | (B37-B42) | (B43-B48) |
| 55 | 401 | 54 | 308 | 53 | 304 | 52 | 300 |
| (C1-C6) | (C7-C12) | (C13-C18) | (C19-C24) | (C25-C30) | (C31-C36) | (C37-C42) | (C43-C48) |
| 51 | 400 | 50 | 307 | 48 | 303 | 47 | F |
| (D1-D6) | (D7-D12) | (D13-D18) | (D19-D24) | (D25-D30) | (D31-D36) | (D37-D42) | (D43-D48) |
| 157 | 310 | 45 | 306 | 44 | F | 43 | 72 |
| (E1-E6) | (E7-E12) | (E13-E18) | (E19-E24) | (E25-E30) | (E31-E36) | (E37-E42) | (E43-E48) |
| 42 | 309 | 41 | F | 40 | 302 | 39 | 71 |
| (F1-F6) | (F7-F12) | (F13-F18) | (F19-F24) | (F25-F30) | (F31-F36) | (F37-F42) | (F43-F48) |
| 38 | F | 37 | 305 | 36 | 301 | 35 | 70 |
| (G1-G6) | (G7-G12) | (G13-G18) | (G19-G24) | (G25-G30) | (G31-G36) | (G37-G42) | (G43-G48) |
| 34 | IgG | 33 | IgG | 32 | IgG | 30 | IgG |
| (H1-H6) | (H7-H12) | (H13-H18) | (H19-H24) | (H25-H30) | (H31-H36) | (H37-H42) | (H43-H48) |
| blank | blank | blank | blank | blank | blank | blank | blank |
| (I1-I6) | (I7-I12) | (I13-I18) | (I19-I24) | (I25-I30) | (I31-I36) | (I37-I42) | (I43-I48) |
| blank | blank | blank | blank | blank | blank | blank | blank |
| (J1-J6) | (J7-J12) | (J13-J18) | (J19-J24) | (J25-J30) | (J31-J36) | (J37-J42) | (J43-J48) |
| blank | blank | blank | blank | blank | blank | blank | blank |
| (K1-K6) | (K7-K12) | (K13-K18) | (K19-K24) | (K25-K30) | (K31-K36) | (K37-K42) | (K43-K48) |
| blank | blank | blank | blank | blank | blank | blank | blank |
| (L1-L6) | (L7-L12) | (L13-L18) | (L19-L24) | (L25-L30) | (L31-L36) | (L37-L42) | (L43-L48) |
| 139 | 148 | 138 | 147 | 137 | 144 | 145 | 162 |
| (M1-M6) | (M7-M12) | (M13-M18) | (M19-M24) | (M25-M30) | (M31-M36) | (M37-M42) | (M43-M48) |
| 29 | 68 | 28 | 124 | 27 | 104 | 26 | 69 |
| (N1-N6) | (N7-N12) | (N13-N18) | (N19-N24) | (N25-N30) | (N31-N36) | (N37-N42) | (N43-N48) |
| 25 | 67 | 24 | 123 | 23 | 103 | 22 | 95 |
| (O1-O6) | (O7-O12) | (O13-O18) | (O19-O24) | (O25-O30) | (O31-O36) | (O37-O42) | (O43-O48) |
| 21 | F | 20 | 122 | 19 | 102 | 18 | 93 |
| (P1-P6) | (P7-P12) | (P13-P18) | (P19-P24) | (P25-P30) | (P31-P36) | (P37-P42) | (P43-P48) |
| 17 | 66 | 16 | F | 15 | 101 | 14 | 91 |
| (Q1-Q6) | (Q7-Q12) | (Q13-Q18) | (Q19-Q24) | (Q25-Q30) | (Q31-Q36) | (Q37-Q42) | (Q43-Q48) |
| 13 | 59 | 12 | 121 | 11 | F | 10 | 90 |
| (R1-R6) | (R7-R12) | (R13-R18) | (R19-R24) | (R25-R30) | (R31-R36) | (R37-R42) | (R43-R48) |
| 8 | 58 | 7 | 120 | 6 | 100 | 5 | F |
| (S1-S6) | (S7-S12) | (S13-S18) | (S19-S24) | (S25-S30) | (S31-S36) | (S37-S42) | (S43-S48) |
| 4 | IgG | 3 | IgG | 2 | IgG | 1 | IgG |
| (T1-T6) | (T7-T12) | (T13-T18) | (T19-T24) | (T25-T30) | (T31-T36) | (T37-T42) | (T43-T48) |
| blank | blank | blank | blank | blank | blank | blank | blank |
| (U1-U6) | (U7-U12) | (U13-U18) | (U19-U24) | (U25-U30) | (U31-U36) | (U37-U42) | (U43-U48) |
| blank | blank | blank | blank | blank | blank | blank | blank |
| (V1-V6) | (V7-V12) | (V13-V18) | (V19-V24) | (V25-V30) | (V31-V36) | (V37-V42) | (V43-V481 |
| blank | blank | blank | blank | blank | blank | blank | blank |
| (W1-W6) | (W7-W12) | (W13-W18) | (W19-W24) | (W25-W30) | (W31-W36) | (W37-W42) | (W43-W48) |
| blank | blank | blank | blank | blank | blank | blank | blank |
| (X1-X6) | (X7-X12) | (X13-X18) | (X19-X24) | (X25-X30) | (X31-X36) | (X37-X42) | (X43-X48) |

### Antibody Binding

Antibody dilutions were made in PBS containing 1% BSA (˜10 mg/ml) and 0.3% Tween-20; the exact concentration for each array is summarized in Table 4. Antibodies were incubated with printed slides for 90-180 min at 4° C. (with the exception of the H3K4me3 monoclonal antibody from Abcam, which was incubated overnight) and washed three times with cold PBS. Arrays were then probed with the appropriate Alexa Fluor 647 conjugated secondary antibody (Invitrogen) for 30-60 min at 4° C., washed three times with cold PBS, and dried. Arrays were then scanned using a Typhoon TR10+ imager (GE Healthcare) at 10 mm resolution using the 526 nm and 670 nm filter sets for the biotin-fluorescein and secondary antibody, respectively. Interactions were quantified using ImageQuant array software (GE Healthcare).

### Protein Expression

The chromatin-associating domains from mouse RAG2 (PHD 387-493), human BPTF (Bromo and PHD domain 2583-2751), and CHD1 (chromodomain 251-467) were C-terminally fused to GST in pGEX-4T. Proteins were heterologously expressed in E. coli and purified by glutathione sepharose affinity chromatography in PBS buffer (50 mMphosphate, 150 mMNaC1, pH 7.6) on an AKTA purifier fast protein liquid chromatography system (GE Healthcare).

### Protein Binding

Prior to binding, arrays were blocked in PBS containing 5% BSA (˜50 mg/mL) and 0.3% Tween-20 for 1 hr at 4° C. to reduce nonspecific binding. Glutathione S-transferase (GST)-tagged protein (w25 mM) in the same buffer was overlaid on each array (200 ml total volume) and incubated in a hybridization chamber at 4° C. overnight. Slides were washed three times with cold PBS. Anti-GST primary antibody was incubated with slides for 90-180 min at 4° C. and washed three times with cold PBS. Arrays were then probed with the Alexa Fluor 647 conjugated anti-rabbit secondary antibody (Invitrogen) for 30-60 min at 4° C., washed three times with cold PBS, and dried. Arrays were then scanned using a Typhoon TR10+ imager (GE Healthcare) at 10 mm resolution using the 526 nm and 670 nm filter sets for the biotin-fluorescein and secondary antibody, respectively. Interactions were quantified using ImageQuant array software (GE Healthcare).

### Statistical Analysis

Briefly, printing of individual spots was evaluated based on the intensity of the fluorescein-biotin cospotted with each peptide. Spots with control intensities of less than 5% of the average intensity for all peptides were labeled as “not spotted” and omitted from subsequent analysis. Data were treated as four individual subarrays to account for small changes in intensity across the slide, each subarray containing all 110 peptides spotted six times. Alexa Fluor 647 intensities (corresponding to a positive interaction) were normalized for all spots by dividing the intensity by the sum of all intensities within a subarray. The six spots for each peptide were averaged (outliers were removed using a Grubbs test) and treated as a single value for a given subarray. The normalized intensities for the four subarrays were used to calculate the mean, and the error is reported as the standard error of the mean. For data displayed as heat maps, mean values were normalized to either the highest calculated value across all peptides or against the peptide for which a given antibody was supposed to interact. Heat maps were created using Java Treeview, and all data were plotted on a scale from 0 to 1 (FIG. 2). Full data sets for all experiments are available at http://www.med.unc.edu/wbstrahl/Arrays/index.htm. Statistical analyses were performed using GraphPad Prism software, Analyses of variance were used to compare interactions, and confidence intervals are reported as 95% (*), 99% (**), or 99.9% (***).

### REFERENCES

1. Walsh, C. T., Garneau-Tsodikova, S., and Gatto, G. J., Jr. (2005). Protein posttranslational modifications: The chemistry of proteome diversifications. Angew. Chem. Int. Ed. Engl. 44, 342-7372.
2. Kouzarides, T. (2007). Chromatin modifications and their function. Cell 128, 693-705.
3. Ruthenburg, A. J., Allis, C. D., and Wysocka, J. (2007). Methylation of lysine 4 on histone H3: intricacy of writing and reading a single epigenetic mark. Mol. Cell. 25, 15-30.
4. Seet, B. T., Dikic, I., Zhou, M. M., and Pawson, T. (2006). Reading protein modifications with interaction domains. Nat. Rev. Mol. Cell. Biol. 7, 473-483.
5, Fuchs, S. M., Laribee, R. N., and Strahl, B. D. (2009). Protein modifications in transcription elongation. Biochim. Biophys. Acta 1789, 26-36.
6. Meek, D. W., and Anderson, C. W. (2009). Posttranslational modification of p53: Cooperative integrators of function. Cold Spring Harb Perspect Biol 1, a000950.
7. Perissi, V., and Rosenfeld, M. G. (2005). Controlling nuclear receptors: The circular logic of cofactor cycles. Nat. Rev. Mol. Cell. Biol. 6, 542-554.
8. Zhang, Y., Jurkowska, R., Soeroes, S., Rajavelu, A., Dhayalan, A., Bock, I., Rathert, P., Brandt, O., Reinhardt, R., Fischle, W., and Jeltsch, A. (2010). Chromatin methylation activity of Dnmt3a and Dnmt3a/3L is guided by interaction of the ADD domain with the histone H3 tail. Nucleic Acids Res. 38, 4246-4253.
9. Kim, T., and Buratowski, S. (2009). Dimethylation of H3K4 by Set1 recruits the Set3 histone deacetylase complex to 50 transcribed regions. Cell 137, 259-272.
10. Hung, T., Binda, O., Champagne, K. S., Kuo, A. J., Johnson, K., Chang, H. Y., Simon, M. D., Kutateladze, T. G., and Gozani, a (2009). ING4 mediates crosstalk between histone H3 K4 trimethylation and H3 acetylation to attenuate cellular transformation. Mol. Cell. 33, 248-256.
11. Taverna, S. D., Ilin, S., Rogers, R. S., Tanny, J. C., Lavender, H., Li, H., Baker, L., Boyle, J., Blair, L. P., Chait, B. T., et al. (2006). Yng1 PHD finger binding to H3 trimethylated at K4 promotes NuA3 HAT activity at K14 of H3 and transcription at a subset of targeted ORFs. Mol. Cell. 24, 785-796,
12. Vermeulen, M., Mulder, K. W., Denissov, S., Pijnappel, W. W., van Schaik, F. M., Varier, R. A., Baltissen, M. P., Stunnenberg, H. G., Mann, M., and Timmers, H. T. (2007). Selective anchoring of TFIID to nucleosomes by trimethylation of histone H3 lysine 4. Cell 131, 58-69.
13. Morris, S. A., Rao, B., Garcia, B. A., Hake, S. B., Diaz, R. L., Shabanowitz, J., Hunt, D. F., Allis, C. D., Lieb, J. D., and Strahl, B. D. (2007). Identification of histone H3 lysine 36 acetylation as a highly conserved histone modification. J. Biol. Chem. 282, 7632-7640.
14. Kasten, M., Szerlong, H., Erdjument-Bromage, H., Tempst, P., Werner, M., and Cairns, B. R. (2004). Tandem bromodomains in the chromatin remodeler RSC recognize acetylated histone H3 Lys14. EMBO J. 23, 1348-1359.
15. Zeng, L., Zhang, Q., Gerona-Navarro, G., Moshkina, N., and Zhou, M. M. (2008). Structural basis of site-specific histone recognition by the bromodomains of human coactivators PCAF and CBP/p300. Structure 16, 643-652.
16. Fischle, W., Wang, Y., and Allis, C. D. (2003). Binary switches and modification cassettes in histone biology and beyond. Nature 425, 475-479.
17. Davies, G. F., Ross, A. R., Arnason, T. G., Juurlink, B. H., and Harkness, T. A. (2010). Troglitazone inhibits histone deacetylase activity in breast cancer cells. Cancer Lett. 288, 236-250.
18. Hayashi-Takanaka, Y., Yamagata, K., Nozaki, N., and Kimura, H. (2009). Visualizing histone modifications in living cells: Spatiotemporal dynamics of H3 phosphorylation during interphase. J. Cell Biol. 187, 781-790.
19. Zhang, X., Zhang, Z., Chen, G., Zhao, M., Wang, D., Zhang, X., Du, Z., Xu, Y., and Yu, X. (2010). FK228 induces mitotic catastrophe in A549 cells by mistargeting chromosomal passenger complex localization through changing centromeric H3K9 hypoacetylation. Acta Biochim. Biophys. Sin. (Shanghai) 42, 677-687.
20. Egelhofer, T. A., Minoda, A., Klugman, S., Lee, K., Kolasinska-Zwierz, P., Alekseyenko, A. A., Cheung, M.-S., Day, D. S., Gadel, S., Gorchakov, A. A., et al. (2010). An assessment of histone-modification antibody quality. Nat. Struct. Mol. Biol., in press. 10.1038/10.1038/nsmb.1972.
21. Ramon-Maiques, S., Kuo, A. J., Carney, D., Matthews, A. G., Oettinger, M. A., Gozani, O., and Yang, W. (2007). The plant homeodomain finger of RAG2 recognizes histone H3 methylated at both lysine-4 and arginine-2. Proc. Natl. Acad. Sci. USA 104, 18993-18998.
22. Varier, R. A., Outchkourov, N. S., de Graaf, P., van Schaik, F. M., Ensing, H. J., Wang, F., Higgins, J. M., Kops, G I, and Timmers, H. M. (2010). A phospho/methyl switch at histone H3 regulates TFIID association with mitotic chromosomes. EMBO J, in press. Published online Oct. 15, 2010. 10.1038/emboj.2010.261.
23. Garske, A. L., Oliver, S. S., Wagner, E. K., Musselman, C. A., LeRoy, G., Garcia, B. A., Kutateladze, T. G., and Denu, J. M. (2010). Combinatorial profiling of chromatin binding modules reveals multisite discrimination. Nat. Chem. Biol. 6, 283-290.
24. Li, H., Ilin, S., Wang, W., Duncan, E. M., Wysocka, J., Allis, C. D., and Patel, D. J. (2006). Molecular basis for site-specific read-out of histone H3K4me3 by the BPTF PHD finger of NURF. Nature 442, 91-95.
25. Flanagan, J. F., Mi, L. Z., Chruszcz, M., Cymborowski, M., Clines, K. L., Kim, Y., Minor, W., Rastinejad, F., and Khorasanizadeh, S. (2005). Double chromodomains cooperate to recognize the methylated histone H3 tail. Nature 438, 1181-1185.
26. Guccione, E., Bassi, C., Casadio, F., Martinato, F., Cesaroni, M., Schuchlautz, H., Lu\ddot{}scher, B., and Amati, B. (2007). Methylation of histone H3R2 by PRMT6 and H3K4 by an MLL complex are mutually exclusive. Nature 449, 933-937.
27. Kirmizis, A., Santos-Rosa, H., Penkett, C. J., Singer, M. A., Vermeulen, M., Mann, M., Ba\ddot{}hler, J., Green, R. D., and Kouzarides, T. (2007). Arginine methylation at histone H3R2 controls deposition of H3K4 trimethylation. Nature 449, 928-932.
28. Bock, I., Dhayalan, A., Kudithipudi, S., Brandt, O., Rathert, P., and Jeltsch, A. (2011). Detailed specificity analysis of antibodies binding to modified histone tails with peptide arrays. Epigenetics 6, 256-263.
29. Bua, D. J., Kuo, A. J., Cheung, P., Liu, C. L., Migliori, V., Espejo, A., Casadio, F., Bassi, C., Amati, B., Bedford, M. T., et al. (2009). Epigenome microarray platform for proteome-wide dissection of chromatin-signaling networks. PLoS ONE 4, e6789.
30. Liu, H., Galka, M., Iberg, A., Wang, Z., Li, L., Voss, C., Jiang, X., Lajoie, G., Huang, Z., Bedford, M. T., and Li, S. S. (2010). Systematic identification of methyllysine-driven interactions for histone and nonhistone targets. J. Proteome Res. 9, 5827-5836.
31. Matthews, A. G., Kuo, A. J., Ramo′ n-Maiques, S., Han, S., Champagne, K. S., Ivanov, D., Gallardo, M., Carney, D., Cheung, P., Ciccone, D. N., et al. (2007). RAG2 PHD finger couples histone H3 lysine 4 trimethylation with V(D)J recombination. Nature 450, 1106-1110.
32. Sims, R. J., 3rd, and Reinberg, D. (2008). Is there a code embedded in proteins that is based on post-translational modifications? Nat. Rev. Mol. Cell. Biol. 9, 815-820.

### Example 2

A microarray platform developed for histone peptides was used to compare the binding properties of human UHRF1 (ubiquitin-like, PHD and RING finger containing 1) tandem Tudor domain (TTD) with other known H3K9 methyl effector proteins, including the chromodomains of the three HP1 isoforms (α, β, γ), the MPP8 chromodomain, and the GLP ankyrin repeats. These peptide microarrays contain a library of 130 unmodified and modified histone peptides representing known single and combinatorial post-translational modifications (PTMs) on the four core histones (H2A, H2B, H3, and H4), including lysine and arginine methylation, lysine acetylation, and serine and threonine phosphorylation (the peptides included peptides listed in Tables 1 and 2). Arrays were spotted 24 times with each histone peptide as described in Rothbart, S. B., et al., Methods in Enzymology 512, 107-135 (2012) and probed with the histidine-tagged (His-tagged) UHRF1 or glutathione S-transferase (GST-tagged) HP1, MPP8, or GLP protein domains. Array analysis revealed that these effector proteins preferentially bound to H3K9 methylated peptides (FIG. 9A). With the exception of the GLP ankyrin repeats, which bound preferentially to H3K9 monomethylation (H3K9me1), these effector proteins had a general preference for H3K9me2 and H3K9me3. No binding was observed for H3K27 methylated peptides, which share a conserved ARKS binding motif with H3K9.

Analyzing the influence of neighboring PTMs on the binding of these effector proteins to H3K9 methylated peptides revealed little influence of lysine acetylation, H3K4me3, or H3R8 methylation (mono-, symmetric, or asymmetric di-methylation), with the exception of the HP1γ chromodomain, whose binding to H3K9me2 and H3K9me3 was partially perturbed by H3R8me2a. In contrast, H3T6p perturbed the binding to H3K9me3 by all tested effector proteins (FIG. 9A). Interestingly, unlike other H3K9 methyl effectors tested, the UHRF1 TTD bound to H3K9me2 and H3K9me3 in the presence of H3S10p (FIG. 9A), consistent with our previous observation using SPOT-array technology (Nady, N. et al., The Journal of biological chemistry 286, 24300-11 (2011)). In-solution peptide pulldown assays verified that the UHRF1 TTD bound H3K9me2 and H3K9me3 regardless of the presence of H3S 10p (FIG. 9B), unlike the MPP8 chromodomain (FIG. 9B) and HP1α. Quantification of this interaction by fluorescence polarization showed the UHRF1 TTD bound to H3K9me3 peptides in the absence or presence of H3S10p with similar affinity (dissociation constants (K_{d}) of 2.0 μM and 2.6 μM, respectively) (FIG. 9C). In contrast, while the MPP8 chromodomain and HP 1α bound to H3K9me3 peptides with K_{d} values of 0.23 μM and 9.0 μM, respectively, binding in the presence of H3S10p was not measurable (n.m.) (FIG. 9C).

### Methods

Materials.

Histone peptides were synthesized, purified, and analyzed as described in Hashimoto, H. et al., Nature 455, 826-9 (2008). Antibodies used in this study: anti-GST (Sigma G7781; 1:1,000), anti-HIS (Santa Cruz sc-8036; 1:200), anti-myc (Millipore 05-419; 1:2,500), anti-Flag (Sigma F1804; 1:5000), anti-streptavidin HRP (Cell Signaling 3999; 1:10,000), anti-UHRF1 (Abeam ab57083; 1:1,000), anti-HP1γ (Cell Signaling 2619; 1:1,000), anti-β-tubulin (Cell Signaling 2146; 1:1,000), anti-H3 (Active Motif 39163; 1:20,000), anti-H3K9me3 (Active Motif 39765; 1:5,000), anti-H3K9me2/S10p (Millipore 05-1354; 1:1,000), anti-H3K9me3/S10p (Millipore 04-809; 1:10,000), anti-H3S10p (Active Motif 39253; 1:5,000), anti-5mC (Diagenode Mab-081; 1:100), anti-cyclin A (Santa Cruz sc-751; 1:2000), anti-cyclin E (Santa Cruz sc-247; 1:1,000). The UHRF1 TTD (human cDNA encoding residues 126-280) was cloned into pET28a-LIC (GenBank accession EF442785) as an N-terminal HIS fusion, expressed in Escherichia coli BL21(DE3) using standard procedures, and purified with Talon resin (ClonTech) according to the manufacturer's protocol. HP 1α (mouse full-length cDNA), HP 1β (mouse full-length cDNA), HP1γ chromodomain (mouse cDNA encoding residues 11-129), and MPP8 chromodomain (human cDNA encoding residues 50-118) were cloned into pGEX-KG (GE Life Sciences). GLP ankyrin repeats (human cDNA encoding residues 734-968) were cloned into pGEX-6P1 (GE Life Sciences). GST fusion proteins were expressed in Escherichia coli BL21(DE3) using standard procedures and purified with GST-bind resin (Novagen) according to the manufacturer's protocol. Full length human UHRF1 was cloned into pCMV-Tag 2 (Agilent) as an N-terminal Flag fusion for mammalian expression. Full length human DNMT1 (a gift from Zhenghe Wang; Case Western) was cloned into pCMV-3Tag (Agilent) as an N-terminal myc fusion for mammalian expression. Point mutations were generated by QuickChange site-directed mutagenesis (Stratagene).

Cell Culture and Manipulation.

HeLa cells (ATCC) were cultured in Minimal Essential Medium (Invitrogen) supplemented with 10% fetal bovine serum (PAA), maintained in a 37° C. incubator with 5% CO₂, and passaged every 2-3 days. E14 and NP95−/− mouse ES cells (a gift from Haruhiko Koseki, RIKEN) were cultured on 0.1% gelatin (Sigma) in Glasgow's Minimal Essential Medium (Invitrogen) supplemented with 15% ES-fetal bovine serum (PAA), 50 units/mL Leukemia Inhibitory Factor (Millipore), 2 mM L-glutamine (Invitrogen), 0.1 mM non-essential amino acids (Invitrogen), 55 μM beta-mercaptoethanol (Invitrogen), 1 mM sodium pyruvate (Invitrogen), 1× penicillin-streptomycin solution P (Invitrogen), maintained in a 37° C. incubator with 5% CO₂, and passaged every 2 days. HeLa cells were synchronized in mitosis with 0.05 μg mL⁻¹ nocodazole for 16 hours. For double thymidine block, HeLa cells were synchronized by treatment with 2 mM thymidine (Sigma) for 16 hours, followed by release for 8 hours, and re-treatment with 2 mM thymidine for 16 hours. Transient transfections were performed using TurboFect (Fermentas) according to the manufacturer's protocol. shRNAs obtained from The RNAi Consortium (TRC) were used following standard TRC Lentivirus production and infection protocols. The indicted concentrations of MG132 (Cayman Chemicals) or 0.05 μg mL⁻¹ nocodazole (Sigma) in DMSO were added during the last 16 hours prior to harvest.

Histone Peptide Microarrays.

Array fabrication and effector protein analysis was performed as described in Rothbart, S. B., et al., Methods in Enzymology 512, 107-135 (2012) and Fuchs, S. M., Krajewski, et al., Current biology 21, 53-58 (2011). Heat maps were generated using Java TreeView.

In-Solution Peptide Pulldowns.

A 50 μL slurry of streptavidin magnetic beads (NEB) was equilibrated in binding buffer containing 50 mM Tris-HCl, pH 8.0, 300 mM NaCl, and 0.1% NP-40 before being saturated with 1 nmole biotinylated peptide for 1 hour at 4° C. with rotation. Unbound peptide was washed with binding buffer, and 100 pmoles of protein in binding buffer supplemented with 0.5% bovine serum albumin (BSA) (w/v) was incubated for 3 hours at 4° C. with rotation. Unbound protein was washed with binding buffer, and bound protein and peptide were eluted from beads by boiling in 1×SDS loading buffer followed by western blot detection. Proteins were detected with anti-HIS (UHRF1) and anti-GST (MPP8) and peptides were detected with anti-streptavidin-HRP.

Fluorescence Polarization.

Peptides for fluorescence polarization (histone H3, residues 1-20) were synthesized as described in Rothbart, S. B., et al., Methods in Enzymology 512, 107-135 (2012) with the addition of 5-carboxyfluorescin (5-FAM) at the N-terminus. Binding assays were performed in 40 μL volume in black flat-bottom 384-well plates (Costar). Protein was titrated with 50 nM peptide in buffer containing 20 mM Tris-HCl, pH 8.0, 250 mM NaCl, 1 mM DTT, and 0.05% NP-40. Following a 20 minute equilibration period at 25° C., plates were read on a POLARstar Omega (BMG Labtech) using a 480 nm excitation filter and 520/530±10 nm emissions filters. Gain settings in the parallel (∥) and perpendicular (⊥) channels were calibrated to a polarization measurement of 100 milli-polarization units (mP) for the fluorescent peptide in the absence of protein. Polarization (P) was determined from raw intensity values of the parallel and perpendicular channels using the equation P═∥−⊥∥+2(⊥) and converted to anisotropy (A) units using the equation A=2P/3−P. Equilibruim dissociation constants (K_{d}) were determined by fitting anisotropy curves to a one-site binding model using GraphPad Prism 5.0.

The foregoing is illustrative of the present invention, and is not to be construed as limiting thereof. The invention is defined by the following claims, with equivalents of the claims to be included therein. All publications, patent applications, patents, patent publications, sequences identified by GenBank and/or SNP accession numbers, and other references cited herein are incorporated by reference in their entireties for the teachings relevant to the sentence and/or paragraph in which the reference is presented.

That which is claimed is:

1. A plurality of synthetic histone peptides, wherein a portion of the synthetic histone peptides comprise at least one post-translational modification, and wherein a portion of the synthetic histone peptides are at least 21 amino acids in length.

2. The plurality of synthetic histone peptides of claim 1, wherein a portion of the synthetic histone peptides comprise at least five post-translational modifications.

3. The plurality of synthetic histone peptides of claim 1, wherein a portion of the synthetic histone peptides comprise a naturally-occurring histone amino acid sequence.

4. The plurality of synthetic histone peptides of claim 1, wherein a portion of the synthetic histone peptides comprise an amino acid sequence that is at least 75% identical to a naturally-occurring histone amino acid sequence.

5. The plurality of synthetic histone peptides of claim 4, wherein the histone amino acid sequence is from a histone selected from the group consisting of H1, H2A, H₂B, H3, H4, H5, or any combination thereof.

6. The plurality of synthetic histone peptides of claim 1, wherein the post-translational modification is selected from the group consisting of phosphorylation, methylation, acetylation, ubiquitination, or any combination thereof.

7. The plurality of synthetic histone peptides of claim 1, wherein the average purity of a majority of the synthetic histone peptides is over about 90%.

8. A peptide array comprising:
a substrate comprising a surface; and
the plurality of synthetic histone peptides of claim 1 immobilized on the substrate surface.

9. The peptide array of claim 8, wherein a portion of the synthetic histone peptides comprise at least five post-translational modifications.

10. The peptide array of claim 8, wherein a portion of the synthetic histone peptides comprise a naturally-occurring histone amino acid sequence.

11. The peptide array of claim 8, wherein a portion of the synthetic histone peptides comprise an amino acid sequence that is at least 75% identical to a naturally-occurring histone amino acid sequence.

12. The peptide array of claim 11, wherein the histone amino acid sequence is from a histone selected from the group consisting of H1, H2A, H2B, H3, H4, H5, or any combination thereof.

13. The peptide array of claim 8, wherein the average purity of a majority of the synthetic histone peptides is over about 90% prior to immobilization on the substrate surface.

14. The peptide array of claim 8, wherein the plurality of synthetic histone peptides are immobilized onto the substrate surface at a high density.

15. The peptide array of claim 11, wherein if the histone amino acid sequence for a synthetic histone peptide in the plurality of synthetic histone peptides is from the N-terminal tail of a histone, then the C-terminus of the synthetic histone peptide is immobilized on the substrate surface and if the histone amino acid sequence for the synthetic histone peptide is from the C-terminal tail of a histone, then the N-terminus of the synthetic histone peptide is immobilized on the substrate surface.

16. The peptide array of claim 8, wherein a synthetic histone peptide in the plurality of synthetic histone peptides comprises one half of a binding pair and the substrate surface comprises the other half of the binding pair.

17. The peptide array of claim 8, further comprising a positive control bound to the substrate surface.

18. The peptide array of claim 17, wherein the positive control is a fluorescent compound.

19. The peptide array of claim 8, wherein a synthetic histone peptide in the plurality of synthetic histone peptides is spotted with a positive control.

20. A method for determining the binding of a protein to a peptide comprising:
providing a peptide array comprising:
a substrate comprising a surface; and
a plurality of synthetic histone peptides immobilized on the substrate surface, wherein a portion of the synthetic histone peptides comprise at least one post-translational modification, and wherein a portion of the synthetic histone peptides are at least 21 amino acids in length;
applying a protein to the peptide array; and
detecting binding of the protein to one or more synthetic histone peptides in the peptide array.

21. A method for detecting the influence of neighboring post-translational modifications on protein binding comprising:
providing a peptide array comprising:
a substrate comprising a surface; and
a plurality of synthetic histone peptides immobilized on the substrate surface, the plurality of synthetic histone peptides comprising peptides with no post-translational modifications, peptides with one post-translational modification, and peptides with more than one post-translational modification, wherein a portion of the synthetic histone peptides are at least 21 amino acids in length;
applying a protein to the peptide array;
detecting binding of the protein to one or more synthetic histone peptides in the peptide array; and
comparing the sequences of the synthetic histone peptides bound to the protein, thereby detecting the influence of neighboring post-translational modifications on protein binding.
